(19) United States
(12) Patent Application Publication
(10) Pub. No.: US 2009/0203015 A1
(43) Pub. Date: Aug. 13, 2009

(54) MULTIPLEX ASSAYS FOR HORMONAL AND GROWTH FACTOR RECEPTORS, AND USES THEREOF

(75) Inventors: Sheng-Yung CHANG, San Francisco, CA (US); Christopher SANTINI, Pleasant Hill, CA (US); Ayuko IVERSON, Sunnyvale, CA (US); Thomas VESS, Garner, NC (US)

Correspondence Address: CELERA CORPORATION, 1401 HARBOR BAY PARKWAY, ALAMEDA, CA, 94502, US

(73) Assignee: CELERA CORPORATION, Alameda, CA (US)

(21) Appl. No.: 12/355,873

(22) Filed: Jan. 19, 2009

Related U.S. Application Data
(60) Provisional application No. 61/065,730, filed on Feb. 13, 2008.

(51) Int. Cl. C12Q 1/68

(52) U.S. Cl. 435/6

(57) ABSTRACT

The present invention provides compositions and methods for simultaneously detecting mRNA expression levels of hormonal receptors, particularly both estrogen receptor (ER) and progesterone receptor (PR), optionally in combination with growth factor receptors, particularly epidermal growth factor receptor ERBB2 (Her-2), and further optionally in combination with control genes, such as the housekeeping genes NUP214 and/or PPIG. Exemplary embodiments of the invention are useful for determining hormonal receptor and/or growth factor receptor status, particular both ER and PR status and optionally also ERBB2 status, such as for assessing or treating breast cancer.

### FIELD OF THE INVENTION

The present invention relates to assaying multiple different hormonal receptors and/or growth factor receptors, particularly for breast cancer assessment and treatment selection. Exemplary embodiments of the invention relate to multiplex assays for simultaneously detecting mRNA levels of multiple different hormonal receptors (particularly estrogen receptor (ER) and progesterone receptor (PR)), optionally together with one or more growth factor receptors (particularly epidermal growth factor receptor ERBB2 (Her-2)), in breast cancer samples.

### BACKGROUND OF THE INVENTION

Estrogen receptor (ER) and progesterone receptor (PR) status in breast cancer patients are factors that are used for therapeutic decisions such as whether or not a patient may benefit from hormonal therapy (Henry and Hayes, Oncologist 2006, 11:541-552) (ESR1 is the gene name for ER, thus ER and ESR1 may be used herein interchangeably; PGR is the gene name for PR, thus PR and PGR may be used herein interchangeably). The American Society of Clinical Oncology (ASCO) recommends routine measurement of ER and PR to identify patients most likely to benefit from hormonal therapy (Harris et al., J Clin Oncol 2007, 25:5287-5312). As an example, studies have shown that patients with ER-positive/PR-negative breast tumors responded less well to hormonal therapy than those with ER-positive/PR-positive breast tumors (Kim et al., Clin Cancer Res 2006, 12: 1013s-1018s and Cui et al., J. Clin Oncol 2005; 23: 7721-7735). In Caucasians, approximately 60-65% of breast cancer cases are ER-positive and PR-positive (ER+/PR+), 15-20% are ER+/PR−, 15-20% are ER−/PR−, and less than 5% are ER−/PR+ (Anderson et al., J Clin Oncol 2001, 19:18-27). Furthermore, the estrogen receptor is the therapeutic target for tamoxifen, a selective estrogen receptor modulator (SERM) that is commonly used in the treatment of breast cancer. ER and PR status in malignant tissue from breast cancer patients provides classification of outcome and clinical benefit for adjuvant endocrine or chemoendocrine therapies such as tamoxifen and aromatase inhibitors. The response rate to tamoxifen treatment has been reported to be markedly decreased in patients with ER+/PR− breast tumors (Cui et al., J Clin Oncol 2005, 23:7721-7735; Arpino et al., J Natl Cancer Inst 2005, 97:1254-1261; and Rakha et al., J Clin Oncol 2007, 25:4772-4778).

Currently, pathologists evaluate the status of these hormone receptors using immunohistochemistry (IHC). A variety of tools have been developed to try to improve the performance of IHC testing for ER and PR, including methods for both manual and image-based scoring of staining results. One example is a semi-quantitative IHC interpretation system, the Allred score, which was developed to grade immunostained slides based upon the percentage and intensity of positively stained tumor cells. However, this approach remains subjective, semi-quantitative, and can be labor-intensive. Moreover, there is a lack of standardization of IHC methods. This has led to inter-laboratory variability and poor reliability for testing of hormonal receptors (Viale et al., J Clin Oncol 2007, 25:3846-3852; Rhodes et al., Am J Clin Pathol 2001, 115:44-58; and Fisher et al., Cancer 2005, 103:164-173) and growth factor receptor Her-2 (Paik et al., J Natl Cancer Inst 2002, 94:852-854 and Reddy et al., Clin Breast Cancer 2006, 7:153-157), including inaccurate measurement of ER status in at least 20% of patients (Diaz and Sneige, Adv Anat Pathol 2005, 12; 10-19; Elledge, Clin Oncol 2006, 24: 1323-1325; Mann et al., J. Clin Oncol 2005, 23; 5148-5154; and Allred et al., Mod Pathol 1998, 11: 155-168). The ASCO 2007 Guideline Update Committee acknowledged that there are “deficits in standardization for ER and PR assays (in particular, IHC), and further efforts at defining reproducibility and accuracy for particular reagents are an important priority” (Harris et al., J Clin Oncol 2007, 25:5287-5312). Various reviews have discussed the issues related to hormone receptor testing for breast cancer (Allred et. al. 1998 (supra), Harvey et. al., J. Clin Oncol 1999, 17: 1474-1481; Diaz and Sneige, 2005 (supra); Mann et. al. 2005 (supra); and Schnitt, J Clin Oncol 2006, 24:1797-1799).

Approximately 25% of tumors in patients with early breast cancer have over-expression of the Her-2 receptor (which may be interchangeably referred to herein as HER2, ERBB2, or epidermal growth factor receptor) or amplification of the Her-2 gene. The disease in these patients is more aggressive, and the risk of recurrence is also higher. Trastuzumab (Herceptin®), a monoclonal antibody directed against the Her-2 receptor, is used to treat these patients (Baselga et al., Oncologist 2006, 11 Suppl 1:4-12, Demonty et al., Eur J Cancer 2007, 43:497-509). Thus, tumor overexpression of Her-2 is used to select women for therapy with trastuzumab. Moreover, high Her-2 expression may be associated with high risk of breast cancer recurrence in women receiving an aromatase inhibitor or tamoxifen as adjuvant therapy (Dowsett et al., J Clin Oncol 2008, 26:1059-65). Therefore, it is useful to determine the expression of Her-2, such as to determine whether an individual has a more aggressive form of breast cancer characterized by over-expression of Her-2 and may benefit from Trastuzumab therapy, for example. New guideline recommendations for Her-2 testing were recently published by ASCO and the College of American Pathologists (Wolff et al., Arch Pathol Lab Med 2007, 131:18-43). Presently, Her-2 status is typically determined using subjective, semi-quantitative IHC assays or quantitative fluorescence in situ hybridization (FISH).

In accordance with conventional terminology, ER, PR, or ERBB2 “status” refers to the relative expression level of each of these genes in a breast tumor sample as compared with the normal range of expression levels in healthy (i.e., non-cancerous) breast samples. The term “positive” with respect to ER, PR, or ERBB2 status indicates that the gene is over-expressed in a breast tumor. In contrast, “negative” indicates that the gene is not over-expressed in a breast tumor.

Molecular assays such as gel-based, semi-quantitative RT-PCR assays (Chevillard et al., Breast Cancer Res Treat 1996, 41:81-89; Tong et al., Anal Biochem 1997, 251:173-177; Hackl et al., Anticancer Res 1998, 18:839-842; Shepard et al., Mod Pathol. 2000, 13:401-406; and Tong et al., Clin Cancer Res 1999, 5:1497-1502) and quantitative assays using real-time RT-PCR and nucleic acid sequence-based amplification (NASBA) technologies (Iwao et al., Cancer 2000, 89:1732-1738; de Cremoux et al., Endocr Relat Cancer 2004, 11:489-495; Labuhn et al., Int J Biol Markers 2006, 21:30-39; and Lamy et al., Clin Chem Lab Med 2006: 44:3-12) have been developed to measure the mRNA level of ER or PR in frozen breast biopsy tissue samples. TaqMan® RT-PCR assays to quantitate ER, PR or HER2 mRNA levels individually in archived formalin-fixed, paraffin-embedded (FFPE) specimens (Cronin et al., Am J Pathol 2004, 164:35-42 and Ma et al., J Clin Oncol 2006, 24:4611-4619) and quantitative PCR assays for HER2 DNA amplification and RT-PCR for overexpression of HER2 mRNA in frozen or FFPE breast tumor specimens (Bìeche et al., Clin Chem. 1999, 45:1148-1156; Millson et al., J Mol Diagn. 2003, 5:184-190; Vinatzer et al., Clin Cancer Res. 2005, 11:8348-8357; Potemski et al., Med Sci Monit. 2006, 12:MT57-61; Kostopoulou et al., Breast. 2007, 16:615-624; Bergqvist et al., Ann Oncol. 2007, 18:845-850; and Barberis et al., Am J Clin Pathol. 2008, 129:563-570) have been reported. Two groups reported the development of amplification-based assays for mRNA levels of ESR1, PGR, and ERBB2 in 2006. Lamy et. al. (Clin Chem Lab Med 2006: 44:3-12) developed a duplex real-time NASBA assay using molecular beacon probes to measure mRNA levels of ESR1 and a housekeeping gene, cyclophilin B (PPIB). The assay format was also applied to PGR with PPIB, or ERBB2 with PPIB. The results were then compared to a duplex quantitation curve to determine the hormonal receptor mRNA level in frozen tissue samples. Labuhn et. al. (Int J Biol Markers. 2006, 21:30-39) developed simplex TaqMan® assays to determine mRNA levels of ESR1, PGR, ERBB1, ERBB2, ERBB3, ERBB4, and housekeeping gene 18S. This assay requires three separate sets of PCR reactions to obtain mRNA levels of ESR1, PGR, and housekeeping gene 18S. However, needing to carry out separate sets of reactions to measure mRNA levels of multiple genes such as both ESR1 and PGR, as well as ERBB2, may typically require extra time, labor, reagents (or other laboratory materials), and/or expense, as well as potentially increasing the likelihood of inaccurate or inconsistent measurements.

Thus, there is a need for a multiplex assay for simultaneously detecting mRNA levels of multiple different hormonal receptors and/or growth factor receptors, such as in a single reaction tube, particularly for breast cancer. Furthermore, there is a particular need for a multiplex assay for simultaneously detecting mRNA levels of ESR1, PGR, and optionally ERBB2.

### SUMMARY OF THE INVENTION

In exemplary embodiments, the present invention provides compositions (e.g., reagents and kits for multiplex assays) and methods for detecting mRNA expression levels of one or more hormonal receptors, particularly both estrogen receptor (ER) and progesterone receptor (PR), optionally in combination with one or more growth factor receptors, particularly epidermal growth factor receptor ERBB2 (interchangeably referred to herein as Her-2 or HER2), and further optionally in combination with one or more control genes, such as the housekeeping genes NUP214 and/or PPIG. In exemplary embodiments, the multiplex assay is carried out in a single reaction tube (or other type of vessel, container, well, etc.) and/or in a one-step process (reagents for detecting multiple different hormonal receptors and/or growth factor receptors are brought into contact with a sample in a single step), thereby providing simultaneous detection of multiple genes (“multiplexing”). Exemplary embodiments of the invention are useful for determining hormonal receptor and/or growth factor receptor status, particular both ER and PR status and optionally also ERBB2 (Her-2) status, such as for diagnosing, prognosing, treating (e.g., selecting a therapeutic agent or treatment strategy), or otherwise assessing breast cancer in an individual.

### BRIEF DESCRIPTIONS OF THE TABLES

Further information regarding each of the tables is provided in “Example One” below.

Table 1 provides a description of sample sets 1, 2, and 3 used for data analyses.

Table 2 provides genes and information about exemplary RT-PCR primers and TaqMan® probes in a mERPR+ or mERPR+HER2 assay (see, e.g., “Example One” below). For example, any of these primers, probes, and reporters can be used in a single-tube, one-step multiplex TaqMan® assay to quantitate mRNA levels of ESR1, PGR, and/or ERBB2, and optionally internal controls (e.g., NUP214 and PPIG), which may be performed on the 7500 system or other system.

Table 3 provides classification of ER status of the discovery and validation sets using the ΔΔC_{T} cutoff-point and clustering methods.

Table 4 provides a summary of the performance of ER classification for the mERPR+HER2 assay.

Table 5 provides classification of PR status of the discovery and validation sets using the ΔΔC_{T} cutoff-point and clustering methods.

Table 6 provides a summary of the performance of PR classification for the mERPR+HER2 assay.

Table 7 provides classification of HER2 overexpression of the discovery and validation sets using the ΔΔC_{T} cutoff-point and clustering methods.

Table 8 provides a summary of the performance of HER2 classification for the mERPR+HER2 assay.

Table 9 provides distributions of immunohistochemistry (IHC) Allred proportion score (PS), intensity score (IS), and total score (TS) for ER and PR of sample set 1 (for both the 7500 and 7900 systems).

Table 10 provides distributions of IHC Allred PS, IS, and TS for ER and PR of sample set 2 (for the 7500 system only).

Table 11 provides distributions of IHC Allred PS, IS, and TS for ER and PR of sample set 3 (for both the 7500 and 7900 systems).

Table 12 provides RNA samples used for determining normalization factor.

Table 13 provides TaqMan® probes for mERPR RT-PCR assay (such as for the 7900 system).

Table 14 provides classification of ER status of the discovery and validation sets using the ΔΔC_{T} cutoff-point and clustering methods (7900 system).

Table 15 provides a summary of the performance of ER classification on the 7900 system.

Table 16 provides classification of PR status of the discovery and validation sets using the ΔΔC_{T} cutoff-point and clustering methods (7900 system).

Table 17 provides a summary of the performance of PR classification on the 7900 system.

Table 18 provides a comparison of ER and PR classification on the 7900 and 7500 systems.

Table 19 provides genes comprising the 14-gene metastasis prognostic panel, as well as 3 endogenous controls (see “Example Two” below). The nucleic acid sequences of each of these 17 genes (as well as the encoded protein sequences) are incorporated herein by reference from the corresponding RefSeq accession number and/or reference citation listed in Table 19 for each gene.

Table 20 provides exemplary fluorescent dyes that may be used in any of the assays disclosed herein.

Table 21 provides an example of parameters used for a clustering analysis.

### DETAILED DESCRIPTION OF EXEMPLARY EMBODIMENTS OF THE INVENTION

The present invention relates to assaying multiple different hormonal receptors and/or growth factor receptors, particularly for breast cancer assessment. Exemplary embodiments of the present invention relate to multiplex assays for detecting mRNA levels of multiple different hormonal receptors (particularly estrogen receptor (ER) and progesterone receptor (PR)), optionally together with one or more growth factor receptors (particularly epidermal growth factor receptor ERBB2, which may be interchangeably referred to herein as Her-2 or HER2), in breast cancer samples, particularly breast tumor tissues preserved by collection methods such as formalin-fixed, paraffin-embedded (FFPE) tumor sections, frozen samples, or other breast cancer specimens. In exemplary embodiments, the multiplex assay is carried out in a single reaction tube (or other type of vessel, container, well, etc.) and/or in a one-step process (reagents for detecting multiple different hormonal receptors and/or growth factor receptors are brought into contact with a sample in a single step), thereby providing simultaneous detection of multiple genes (“multiplexing”). In various exemplary embodiments, the multiplex assay is a TaqMan® assay. “Multiplex” is used herein in accordance with conventional usage of this term in the art; e.g., a “multiplex” assay is an assay designed to detect, measure, analyze, or otherwise assess multiple targets, such as to detect mRNA expression levels of multiple genes. The term “mERPR+” may be used herein to refer to an exemplary assay for detecting expression of ER and PR, and the term “mERPR+HER2” may be used herein to refer to an exemplary assay for detecting expression of ER, PR, and HER2.

Exemplary assays of the invention may include assays for multiple different hormonal receptors, assays for multiple different growth factor receptors, and assays for one or more hormonal receptors in combination with one or more growth factor receptors (such as assays for multiple hormonal receptors in combination with a growth factor receptor). Any of these assays can optionally further include assays for control genes, such as to normalize mRNA expression levels. Exemplary control genes include housekeeping genes (HSKs) such as NUP214 and PPIG. Exemplary assays of the invention, such as multiplex TaqMan® assays, enable quantitative detection of mRNA levels in degraded RNA, such as in small amounts of RNA extracted from FFPE slides from breast cancer patients, in a single tube using one-step RT-PCR.

Specific exemplary embodiments of the invention include, but are not limited to, compositions (e.g., reagents and kits for multiplex assays such as TaqMan® assays) and methods for simultaneously (e.g., in a multiplex assay) detecting mRNA of the following combinations of genes (these combinations can comprise or consist of the listed genes):

1) ESR1 and PGR

2) ESR1, PGR, and ERBB2

3) ESR1, PGR, NUP214, and PPIG

4) ESR1, PGR, ERBB2, NUP214, and PPIG

5) ESR1, PGR, and NUP214

6) ESR1, PGR, and PPIG

7) ESR1, PGR, ERBB2, and NUP214

8) ESR1, PGR, ERBB2, and PPIG

9) ESR1, PGR, and at least one control gene

10) ESR1, PGR, ERBB2, and at least one control gene

11) ESR1, PGR, and a growth factor receptor gene

12) ESR1 and ERBB2

13) PGR and ERBB2

14) ESR1, ERBB2, and optionally at least one control gene (which may include NUP214 and/or PPIG)

15) PGR, ERBB2, and optionally at least one control gene (which may include NUP214 and/or PPIG)

16) ERBB2 and a hormonal receptor gene, and optionally at least one control gene (which may include NUP214 and/or PPIG)

17) ESR1 and a growth factor receptor gene, and optionally at least one control gene (which may include NUP214 and/or PPIG)

18) PGR and a growth factor receptor gene, and optionally at least one control gene (which may include NUP214 and/or PPIG)

19) ESR1, PGR, a growth factor receptor gene, and optionally at least one control gene (which may include NUP214 and/or PPIG)

20) ESR1 and/or PGR, ERBB2, and at least one other growth factor receptor gene (other than ERBB2), and optionally at least one control gene (which may include NUP214 and/or PPIG)

21) ESR1 and/or PGR, ERBB2, and at least one other hormonal receptor gene (other than ESR1 and PGR), and optionally at least one control gene (which may include NUP214 and/or PPIG)

22) ESR1 and/or PGR, and at least one other gene of interest, and optionally at least one control gene (which may include NUP214 and/or PPIG)

23) ESR1 and/or PGR, ERBB2, and at least one other gene of interest, and optionally at least one control gene (which may include NUP214 and/or PPIG)

Thus, in certain embodiments, for example, the multiplex assay quantitatively detects ESR1 and PGR mRNA levels. In further exemplary embodiments, the multiplex assay quantitatively detects ESR1 and PGR mRNA levels in combination with the HSKs NUP214 and/or PPIG. In further exemplary embodiments, the multiplex assay quantitatively detects ESR1, PGR, and ERBB2 (Her-2) mRNA levels. In yet further exemplary embodiments, the multiplex assay quantitatively detects ESR1, PGR, and ERBB2 mRNA levels in combination with the HSKs NUP214 and/or PPIG.

Control genes can be used to normalize expression data, such as according to the method described in J. Vandesompele, K. De Preter et al., Genome Biol 3(7): Research 0034.1-0034.11 (Epub 2002). The term “control gene”, as used herein, refers to any gene used for normalizing gene expression. “Housekeeping gene” (“HSK”), generally refers to a gene that is constitutively expressed and may be involved in basic functions needed for the sustenance of a cell (in accordance with the typical definition in the art of “housekeeping gene”). Housekeeping genes are an example of a type of control gene.

Detection of the mRNA levels of HSKs such as NUP214 and PPIG (or other control genes), although not necessary, is useful for normalizing ESR1, PGR, and/or ERBB2 mRNA levels (and/or the mRNA levels of other hormonal receptors or growth factor receptors). When the mRNA levels of multiple different HSKs (or other control genes) are detected, such as both NUP214 and PPIG, the average mRNA expression levels (or other combination of mRNA levels) can be used to normalize ESR1, PGR, and/or ERBB2 mRNA levels. For example, a Ct representing the average of the Cts obtained from amplification of multiple control genes (Ct_{EC}) can be used to minimize the risk of normalization bias that may occur if only one control gene were used (T. Suzuki, P J Higgins et al., 2000, Biotechniques 29:332-337). The adjusted expression level of the gene(s) of interest may optionally be further normalized to a calibrator reference RNA pool such as ref RNA (Universal Human Reference RNA, Stratagene, La Jolla, Calif.), or other control sample, such as to standardize expression results obtained from different instruments.

Certain exemplary embodiments of the invention provide a 4-plex assay for quantitating mRNA levels of ESR1, PGR, NUP214, and PPIG (hereinafter referred to as the “4-plex assay”). Other exemplary embodiments of the invention provide a 5-plex assay for quantitating these four genes plus ERBB2 (hereinafter referred to as the “5-plex assay”).

In an exemplary 4-plex assay, FAM-labeled probes (e.g., TaqMan® probes) and TRE-labeled probes (e.g., TaqMan® probes) can be designed to detect ESR1 and PGR amplicons, respectively (alternatively, VIC-labeled probes can be used to detect PGR). VIC-labeled probes (e.g., TaqMan® probes) can be designed to detect amplicons from two HSKs (e.g., NUP214 and PPIG) (alternatively, TET-labeled, NED-labeled, and/or TRE-labeled probes can be used to detect the HSKs; however, it is preferable that TRE-labeled probes be used to detect the HSKs only if VIC-labeled probes or another label are used to detect PGR rather than TRE-labeled probes). In exemplary embodiments, the mRNA levels of ESR1, PGR, and 2 HSKs can be detected simultaneously in a multiplex reaction with 8 primers and 4 probes (e.g., TaqMan® probes). In certain exemplary embodiments of the 4-plex assay, three different fluorescent reporters are used (a different label for each of ESR1 and PGR, plus the same label for each of the 2 HSKs).

In an exemplary 5-plex assay, probes and primers for detecting ERBB2 are added to the 4-plex assay described in the preceding paragraph. For example, PHO-labeled probes (e.g., TaqMan® probes) can be designed to detect ERBB2 amplicon (alternatively, TET-labeled or NED-labeled probes can be used to detect ERBB2, preferably if these labels are not used to detect the HSKs). In exemplary embodiments, the addition of ERBB2 primers and probes (e.g., PHO-labeled probes) enables the simultaneous detection of ESR1, PGR, ERBB2, and 2 HSK amplicons in a multiplex reaction with 10 primers and 5 probes (e.g., TaqMan® probes). In certain exemplary embodiments of the 5-plex assay, four different fluorescent reporters are used (a different label for each of ESR1, PGR, and ERBB2, plus the same label for each of the 2 HSKs). Three probes (e.g., TaqMan® probes) labeled with three different fluorescent reporters (e.g., FAM, TRE, and PHO) are designed to detect PCR product from ESR1, PGR, and ERBB2 (Table 2). Two probes (e.g., TaqMan® probes), which may be labeled with the same 4^{th} fluorescent reporter (e.g., VIC) such as to minimize the types of fluorescent reporters used in the assay, are designed to detect PCR product from two HSKs (e.g., NUP214 and PPIG).

Any combinations of fluorescent reporters (e.g., three different reporters in the 4-plex assay or four different reporters in the 5-plex assay), preferably having minimal crosstalk and that are compatible with a real-time PCR instrument, can be used for the exemplary assays (e.g., TaqMan® assays), such as the 4-plex or 5-plex assays. Probes (e.g., TaqMan® probes) with minor-groove binder (MGB) can be used to increase the melting temperature of the probes (particularly for short probes), and probes can optionally be labeled with non-fluorescent quencher (NFQ) at the 3′ end of the probe. ROX can be used as a passive reference dye. Human Universal Reference RNA (Stratagene) and NTC (no template control) may also be used in an experiment.

Table 20 provides examples of fluorescent dyes that may be used in any of the assays disclosed herein. Any other fluorescent dyes known in the art may be used as well. Furthermore, other labels besides fluorescent dyes may be used. Any labels, fluorescent or otherwise, that are useful for detecting gene expression may be used. Furthermore, if expression detection of other genes of interest (such as genes other than ER, PR, and ERBB2) and/or other control genes (such as control genes other than the housekeeping genes NUP214 and PPIG) is added to an assay, then other fluorescent dyes (or other types of labels) including, but not limited to, any of the fluorescent dyes listed in Table 20 may be used to label the expression products (e.g., mRNA) of these other genes. As an example, five different fluorescent dyes could be used to detect expression of four genes of interest (e.g., ER, PR, ERBB2, plus any other gene of interest, such as any of the genes disclosed in the “Other Genes of Interest” section below) and one or more control genes (e.g., the two housekeeping genes NUP214 and PPIG), using a different dye for each of the four genes of interest and a 5^{th} dye to detect each of the control genes.

In exemplary embodiments, the invention provides a quantitative method to detect ESR1, PGR, and (optionally) ERBB2 expression levels in a single tube, thus providing a high-throughput multiplex RT-PCR platform useful for clinical laboratory testing, for example. The exemplary assays of the invention provide reliable quantitative measurements of hormone receptor levels (and, optionally, growth factor receptor levels) in breast cancer patients that can aid medical practitioners in making informed treatment decisions. For example, exemplary embodiments of the invention provide assay results for ER, PR, and optionally ERBB2 (Her-2) receptor status, which medical practitioners can use, for example, to determine whether a patient (e.g., an individual having breast cancer) may benefit from hormonal therapy (e.g., selective estrogen receptor modulators such as tamoxifen), aromatase inhibitors, and/or Trastuzumab (Herceptin®), as well as other treatments and therapeutic agents.

Accordingly, a medical practitioner can use the compositions and methods of the invention to determine whether an individual having breast cancer is likely to respond positively or otherwise benefit from a particular treatment, or whether an individual is unlikely to respond or benefit from a particular treatment or is likely to suffer adverse side effects, thereby enabling a medical practitioner to select a treatment or otherwise implement a treatment strategy for an individual. Treatments can include, but are not limited to, Trastuzumab (Herceptin®) and other therapeutic agents that target the Her-2 receptor, such as other antibodies or small molecule compounds, hormonal therapies such as selective estrogen receptor modulators (SERMs) (e.g., tamoxifen), aromatase inhibitors, as well as other treatments and therapeutic agents.

An aspect of the invention relates to methods of determining ER and PR status and, optionally, ERBB2 status, in a breast cancer patient, comprising measuring mRNA expression of the genes known as ESR1, PGR, and (optionally) ERBB2 (Her-2), and determining ER and PR status and, optionally, ERBB2 (Her-2) status based on mRNA expression levels of these genes.

Another aspect of the invention relates to methods of determining ER and PR status and, optionally, ERBB2 status, in a breast cancer patient, in which measurements of ESR1, PGR, and (optionally) ERBB2 mRNA expression are normalized against the mRNA expression of one or more control genes. In certain aspects of the invention, the control genes comprise at least one of NUP214 and PPIG.

Another aspect of the invention relates to methods of determining ER and PR status and, optionally, ERBB2 status, in a breast cancer patient, in which ESR1, PGR, and (optionally) ERBB2 mRNA is reverse transcribed to cDNA and detected by polymerase chain reaction amplification.

Another aspect of the invention relates to methods of determining ER and PR status and, optionally, ERBB2 status, in a breast cancer patient, in which ESR1, PGR, and (optionally) ERBB2 mRNA is reverse transcribed to cDNA and amplified by the primers for each of these genes as presented in Table 2, SEQ ID NOS: 1-2, 4-5, and 7-8. Optionally, NUP214 and/or PPIG mRNA can also be amplified in combination with ESR1, PGR, and/or ERBB2 using the primers for NUP214 and PPIG as presented in Table 2, SEQ ID NOS:10-11 and 13-14.

In another aspect of the invention, ESR1, PGR, and/or ERBB2 nucleic acid is contacted with a probe for each of these genes as presented in Table 2, SEQ ID NOS:3, 6, and 9. Optionally, NUP214 and/or PPIG nucleic acid is also contacted with a probe for each of these genes as presented in Table 2, SEQ ID NOS:12 and 15.

In certain embodiments of the invention, any of ESR1, PGR, and/or ERBB2 (and optionally NUP214 and/or PPIG) are amplified by primers for each of these genes as presented in Table 2, SEQ ID NOS: 1-2, 4-5, and 7-8 (and optionally SEQ ID NOS: 10-11 and 13-14 for NUP214 and PPIG), and are also contacted with a probe for each of these genes as presented in Table 2, SEQ ID NOS:3, 6, and 9 (and optionally SEQ ID NOS: 12 and 15 for NUP214 and PPIG).

Another aspect of the invention relates to methods of determining ER and PR status and, optionally, ERBB2 status, in a breast cancer patient, in which ESR1, PGR, and (optionally) ERBB2 mRNA expression is detected by a microarray.

Thus, exemplary embodiments of the invention provide, for example, multiplex assays for detecting mRNA levels of multiple different hormonal receptors (particularly ESR1 and PGR) and/or growth factor receptors (particularly ERBB2), methods of determining expression levels of these genes in a test sample, methods of determining hormonal receptor and/or growth factor receptor status (particularly ER, PR, and/or ERBB2 status), and methods of using these assays and methods, such as to diagnose or prognose breast cancer or to select a therapeutic agent or treatment strategy for breast cancer (e.g., determine whether an individual may benefit from hormonal therapy and/or Trastuzumab (Herceptin®) treatment).

Representative Gene Information

Expression profiling of the ESR1, PGR, and ERBB2 genes allows for determining ER, PR, and ERBB2 status, respectively, such as for breast cancer assessment. Control genes such as the NUP214 and PPIG housekeeping genes are useful for normalizing ESR1, PGR, and ERBB2 mRNA levels (and mRNA levels of other genes as well). The ESR1, PGR, ERBB2, NUP214, and PPIG genes are known in the art. The following provides information about these genes and the encoded proteins, including a reference sequence (RefSeq accession number) (obtained from the National Center for Biotechnology Information (NCBI) of the National Institutes of Health/National Library of Medicine) that identifies an exemplary transcript sequence of each described gene, as well as a citation for a reference that published the nucleotide sequence of each RefSeq. The nucleic acid and encoded protein sequences disclosed in each of these RefSeq accession numbers and reference citations are incorporated herein by reference.

The ESR1 (estrogen receptor) gene, an exemplary sequence of which is provided by reference sequence NM_{—}000125 (SEQ ID NO:16), and disclosed in Greene G L, Gilna P, et al., “Sequence and expression of human estrogen receptor complementary DNA”, Science. 1986, 231(4742):1150-1154. Three other ESR1 sequence variants are provided as reference sequences AF258449 (SEQ ID NO: 17), AF258450 (SEQ ID NO:18), and AF258451 (SEQ ID NO:19). Said reference sequences and reference citation are herein incorporated by reference in their entirety.

The PGR (progesterone receptor) gene, an exemplary sequence of which is provided by reference sequence NM_{—}000926 (SEQ ID NO:20), and disclosed in Misrahi M, Atger M, et al., “Complete amino acid sequence of the human progesterone receptor deduced from cloned cDNA”, Biochem Biophys Res Commun. 1987, 143(2):740-748. Three other PGR sequence variants are provided as reference sequences AB085683 (SEQ ID NO:21), AB085844 (SEQ ID NO:22), and AB085845 (SEQ ID NO:23). Said reference sequences and reference citation are herein incorporated by reference in their entirety.

The ERBB2 gene (a member of the epidermal growth factor (EGF) receptor family of receptor tyrosine kinases), an exemplary sequence of which is provided by reference sequences NM_{—}004448 (SEQ ID NO:24) and NM_{—}001005862 (SEQ ID NO:25), and disclosed in Coussens L, Yang-Feng T L, et al., “Tyrosine kinase receptor with extensive homology to EGF receptor shares chromosomal location with neu oncogene”, Science. 1985, 230(4730), 1132-1139. Said reference sequences and reference citation are herein incorporated by reference in their entirety.

The NUP214 (nucleoporin 214 kDa) gene, an exemplary sequence of which is provided by reference sequence NM_{—}005085 (SEQ ID NO:26), and disclosed in Graux, C., Cools, J. et al., 2004, Nat. Genet. 36 (10), 1084-1089. Said reference sequence and reference citation are herein incorporated by reference in their entirety.

The PPIG (peptidylprolyl isomerase G) gene, an exemplary sequence of which is provided by reference sequence NM_{—}004792 (SEQ ID NO:27), and disclosed in Lin, C. L., Leu, S. et al., 2004, Biochem. Biophys. Res. Commun. 321 (3), 638-647. Said reference sequence and reference citation are herein incorporated by reference in their entirety.

The ESR1, PGR, ERBB2, NUP214, and PPIG genes, and expression products thereof (e.g., mRNA and, in certain embodiments, protein), may be referred to in the present description by such terms/phrases as “genes”, “genes of the present invention”, “genes disclosed herein”, “gene sequences of the present invention”, or “gene sequences disclosed herein”, and similar terms/phrases. Thus, references herein to “genes” typically may also include gene expression products such as mRNA (as well as protein, depending on the embodiment, which will be apparent to one of ordinary skill in the art), and are not necessarily limited to the genomic DNA sequence of a gene, for example.

Table 2 provides exemplary primer sets and exemplary probes that can be used to detect each gene. Based on the reference sequences for each gene, such as the reference sequences provided herein, other reagents (e.g., other primers and/or probes) may be designed to detect these genes, and reagents can be designed to detect any and all variants of each gene. Thus, the present invention provides for expression profiling of all known transcript variants of the genes disclosed herein.

Exemplary Combinations Comprising Additional Genes

The exemplary assays provided by the invention can also complement, and can be used in conjunction with, other genes and other breast cancer assays such as prognosis signature assays that predict the risk of breast cancer metastasis, for example. An example of such an assay for predicting the risk of breast cancer metastasis is the 14-gene prognostic assay described in U.S. patent application Ser. No. 12/012,530, Kit Lau et al., filed Jan. 31, 2008, incorporated herein by reference in its entirety. An example of an assay in which ESR1, PGR, and ERBB2 are combined with this 14-gene prognostic assay along with three control genes (housekeeping genes), for a total of 20 genes that are assayed in five multiplex assays, is described in Example Two below. In Example Two, the 14 genes of interest are as follows (these 14 genes are collectively referred to herein as the “14-gene signature”): CENPA, PKMYT1, MELK, MYBL2, BUB1, RACGAP1, TK1, UBE2S, C16orf61 (DC13), RFC4, PRR11 (FLJ11029), DIAPH3, ORC6L, and CCNB1, and the 3 control genes (housekeeping genes) are PPIG, NUP214, and SLU7. These 14 genes of interest and 3 control genes are described in U.S. patent application Ser. No. 12/012,530, Kit Lau et al., filed Jan. 31, 2008, which is incorporated herein by reference in its entirety, and are also shown in Table 19 of the instant application (Table 19 of the instant application corresponds with Table 2 of U.S. patent application Ser. No. 12/012,530). Any combination of these 14 genes of interest and 3 control genes can be combined with any combination of ESR1, PGR, and/or ERBB2. For example, certain exemplary embodiments of the invention include assays for measuring mRNA expression levels of the following combinations of genes (these combinations can comprise or consist of the listed genes):

1) ESR1, PGR, and the 14-gene signature

2) ESR1, PGR, ERBB2, and the 14-gene signature

3) ESR1, PGR, the 14-gene signature, and at least one control gene (which may include, but is not limited to, any combination of, or all of, NUP214, PPIG, and/or SLU7)

4) ESR1, PGR, ERBB2, the 14-gene signature, and at least one control gene (which may include, but is not limited to, any combination of, or all of, NUP214, PPIG, and/or SLU7)

5) ESR1, PGR, and at least one of the genes of the 14-gene signature

6) ESR1, PGR, ERBB2, and at least one of the genes of the 14-gene signature

7) ESR1, PGR, at least one of the genes of the 14-gene signature, and at least one control gene (which may include, but is not limited to, any combination of, or all of, NUP214, PPIG, and/or SLU7)

8) ESR1, PGR, ERBB2, at least one of the genes of the 14-gene signature, and at least one control gene (which may include, but is not limited to, any combination of, or all of, NUP214, PPIG, and/or SLU7)

9) any combination of at least one, two, or all three of ESR1, PGR, and/or ERBB2, in combination with at least one of the genes of the 14-gene signature, and optionally further in combination with at least one control gene (which may include, but is not limited to, any combination of, or all of, NUP214, PPIG, and/or SLU7)

When combined with other genes or other breast cancer assays (such as the 14-gene prognostic assay for predicting the risk of breast cancer metastasis), ESR1, PGR, and/or ERBB2 can be assayed in a single reaction tube or in separate reaction tubes. For example, in the exemplary assay described in Example Two below, ESR1, PGR, and ERBB2 are combined with the 14-gene prognostic assay along with three HSKs, for a total of 20 genes that are assayed in five multiplex assays. In this exemplary assay, ESR1, PGR, and ERBB2 can each be assayed in a separate one of the five multiplex assays (e.g., in different reaction tubes), or all three of these genes can be assayed in the same reaction tube, or any combination of two of these three genes can be assayed in a single reaction tube while the third gene is assayed in a separate reaction tube.

Those skilled in the art will readily recognize that nucleic acid molecules may be double-stranded molecules and that reference to a particular sequence of one strand refers, as well, to the corresponding site on a complementary strand. In defining a nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular nucleotide sequence. Probes and primers may be designed to hybridize to either strand and gene expression profiling methods disclosed herein may generally target either strand.

Other Genes of Interest

The assays disclosed herein can be designed to detect any other genes of interest (in addition to ESR1, PGR, and/or ERBB2), as well as any alternative splice variants of these genes of interest. For example, a 5^{th} fluorescent dye can be added to the mERPR+HER2 assay for detection of an additional gene of interest. Any genes that are useful for cancer assessment, especially assessment of breast cancer, are examples of genes of interest which can be detected by an assay disclosed herein. Examples of genes of interest include, but are not limited to, other growth factor receptors (in addition to ERBB2) and other hormonal receptors (in addition to ESR1 and PGR), as well as any alternative splice variants of growth factor receptors and hormonal receptors. Examples of other growth factor receptors include, but are not limited to, EGFR (also known as ERBB1 or HER1), ERBB3 (also known as HER3), and ERBB4 (also known as HER4). Examples of other hormonal receptors include, but are not limited to, ESR2 and androgen receptor (AR). Other genes of interest include genes associated with treatment response, such as genes associated with response to hormonal treatments such as tamoxifen. Examples of tamoxifen response related genes include, but not limited to, BCL2, FOS, IGFBP4, MET, SNCG (Vendrell J A, et al., Breast Cancer Res. 2008, 10:R88), NCOR1 (Girault I et al., Clin Cancer Res. 2003, 9:1259-1266), CGA (Bieche I et al., Cancer Res. 2001, 61:1652-1658), C6orf66, TIMELESS, PTPLB, FAM100B (Tozlu-Kara et al., J Mol Endocrinol. 2007, 39:305-318), HOXB13, IL-17BR (Goetz et al., J. Clin Cancer Res. 2006, 12:2080-2087), CYP2D6 (Goetz et al., Clin Pharmacol Ther. 2008, 83:160-166), AKT1, AKT2, BCAR1, BCAR3, EGFR, ERBB2, GRB7, SRC, TLE3, TRERF1 (van Agthoven et al. J Clin Oncol. 2008), and ESRRG (Riggins et al., Cancer Res. 2008, 68:8908-8917). Any of these genes are examples of other genes of interest which can be detected by an assay disclosed herein (e.g., by using reagents labeled with a dye that is different than the dyes used to detect ESR1, PGR, and/or ERBB2). Furthermore, any of the 14 genes listed in Table 19 are examples of other genes of interest which can be detected by an assay disclosed herein.

Tumor Tissue Source and RNA Extraction

In exemplary embodiments of the invention, nucleic acids are extracted from a sample taken from an individual afflicted with breast cancer. The sample may be collected in any clinically acceptable manner, typically such that gene-specific polynucleotides (e.g., mRNA) are preserved. The nucleic acids so obtained from the sample may then be analyzed further. Target polynucleotides may be analyzed directly in whole nucleic acids (e.g., genomic DNA or total RNA) or, optionally, target polynucleotides may be enriched and/or amplified from among whole nucleic acids. For example, pairs of oligonucleotides specific for a gene (e.g., the ESR1, PGR, ERBB2, NUP214, and/or PPIG genes; Table 2 provides exemplary primer pairs for amplifying these genes) may be used to amplify specific mRNA(s) in the sample. The amount of each message can be determined, or profiled, and a determination of gene status (for example) can be made, such as for breast cancer diagnostic, prognostic, or treatment selection purposes. Alternatively, mRNA or nucleic acids derived therefrom (e.g., cDNA, amplified DNA, or enriched RNA) may be labeled distinguishably from standard or control polynucleotide molecules, and both may be simultaneously or independently hybridized to a microarray (or other composition) comprising probes for detecting some or all of the hormonal receptor and/or growth factor receptor genes described herein. Alternatively, mRNA or nucleic acids derived therefrom may be labeled with the same label as the standard or control polynucleotide molecules, wherein the intensity of hybridization of each at a particular probe is compared.

A sample may comprise any clinically relevant tissue sample, such as a formalin-fixed paraffin-embedded (FFPE) sample, frozen sample, tumor biopsy or fine needle aspirate, or a sample of bodily fluid containing tumor cells such as blood, plasma, serum, lymph, ascitic or cystic fluid, urine, or nipple exudate. Exemplary embodiments of the invention are particularly well-suited for detecting mRNA levels from degraded samples or samples with small amounts of RNA, such as small samples of RNA extracted from FFPE samples or other tumor biopsy specimens.

Methods for preparing total and poly (A)+RNA are well known and are described generally in Sambrook et al., MOLECULAR CLONING—A LABORATORY MANUAL (2nd ed.), Vols. 1-3, Cold Spring Harbor Laboratory, Cold Spring Harbor, N.Y. (1989) and Ausubel et al., Current Protocols in Molecular Biology vol. 2, Current Protocols Publishing, New York (1994). RNA may be isolated from tumor cells by any procedures well-known in the art, generally involving lysis of the cells and denaturation of the proteins contained therein.

As an example of preparing RNA from tissue samples, RNA may also be isolated from FFPE tissues using techniques well known in the art. Commercial kits for this purpose may be obtained, e.g., from Zymo Research, Ambion, Qiagen, or Stratagene. An exemplary method of isolating total RNA from FFPE tissue, according to the method of the Pinpoint Slide RNA Isolation System (Zymo Research, Orange, Calif.) is as follows. Briefly, the solution obtained from the Zymo kit is applied over the selected FFPE tissue region of interest and allowed to dry. The embedded tissue is then removed from the slide and placed in a centrifuge tube with proteinase K. The tissue is incubated for several hours, then the cell lysate is centrifuged and the supernatant transferred to another tube. RNA is extracted from the lysate by means of a guanidinium thiocynate/β mercaptoethanol solution, to which ethanol is added and mixed. Sample is applied to a spin column, and spun for one minute. The column is washed with buffer containing ethanol and Tris/EDTA. dNase is added to the column, and incubated. RNA is eluted from the column by adding heated rNase-free water to the column and centrifuging. Pure total RNA is present in the eluate.

Additional steps may be employed to remove contaminating DNA, such as the addition of dNase to the spin column, described above. Cell lysis may be accomplished with a nonionic detergent, followed by micro-centrifugation to remove the nuclei and hence the bulk of the cellular DNA. In one embodiment, RNA is extracted from cells of the various types of interest by cell lysis in the presence of guanidinium thiocyanate, followed by CsCl centrifugation to separate the RNA from DNA (Chirgwin et al., Biochemistry 18:5294-5299 (1979)). Poly(A)+RNA is selected with oligo-dT cellulose (see Sambrook et al., MOLECULAR CLONING—A LABORATORY MANUAL (^{2N}D ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types it may be desirable to add a protein denaturation/digestion step to the protocol.

For certain applications, it is desirable to preferentially enrich mRNA with respect to other cellular RNAs extracted from cells, such as transfer RNA (tRNA) and ribosomal RNA (rRNA). Most mRNAs contain poly(A) tails at their 3′ ends. This allows for enrichment by affinity chromatography; for example, using oligo(dT) or poly(U) coupled to a solid support, such as cellulose or Sephadex™ (see Ausubel et al., CURRENT PROTOCOLS IN MOLECULAR BIOLOGY, vol. 2, Current Protocols Publishing, New York (1994)). After being bound in this manner, poly(A)+mRNA is eluted from the affinity column using 2 mM EDTA/0.1% SDS.

The sample of RNA can comprise a plurality of different mRNA molecules, each mRNA molecules having a different nucleotide sequence. In a specific embodiment, the mRNA molecules of the RNA sample comprise mRNA expressed by one or more of the ESR1, PGR, ERBB2, NUP214, and PPIG genes, particularly mRNA expressed by ESR1 and PGR and optionally ERBB2. In a further specific embodiment, total RNA or mRNA from cells is used in the methods of the invention. The source of the RNA can be cells from a tumor cell, for example, particularly a breast tumor cell. In specific embodiments, the method of the invention is used with a sample containing total mRNA or total RNA from 1×10⁶ cells or fewer.

Reagents for Measuring Gene Expression

The present invention provides nucleic acid molecules that can be used in gene expression profiling and in assessing breast cancer. Exemplary nucleic acid molecules that can be used as primers and probes in various assays of the invention are shown in Table 2 (but primers and probes for these genes are not limited to these disclosed oligonucleotides).

As indicated in Table 2:

ESR 1 mRNA can be reverse-transcribed and amplified with SEQ ID NO: 1 as the forward primer and SEQ ID NO:2 as the reverse primer, and SEQ ID NO:3 can be used as a probe in a TaqMan® or other assay.

PGR mRNA can be reverse-transcribed and amplified with SEQ ID NO:4 as the forward primer and SEQ ID NO:5 as the reverse primer, and SEQ ID NO:6 can be used as a probe in a TaqMan® or other assay.

ERBB2 mRNA can be reverse-transcribed and amplified with SEQ ID NO:7 as the forward primer and SEQ ID NO:8 as the reverse primer, and SEQ ID NO:9 can be used as a probe in a TaqMan® or other assay.

NUP214 mRNA can be reverse-transcribed and amplified with SEQ ID NO:10 as the forward primer and SEQ ID NO:11 as the reverse primer, and SEQ ID NO:12 can be used as a probe in a TaqMan® or other assay.

PPIG mRNA can be reverse-transcribed and amplified with SEQ ID NO:13 as the forward primer and SEQ ID NO:14 as the reverse primer, and SEQ ID NO:15 can be used as a probe in a TaqMan® or other assay.

Alternative primers and/or probes that can be used in the assays described herein can be designed and synthesized.

In a specific aspect of the invention, the oligonucleotide sequences disclosed in Table 2 can be used as gene expression profiling reagents. As used herein, a “gene expression profiling reagent” is a reagent that is specifically useful in the process of amplifying and/or detecting the nucleotide sequence of a specific target gene, regardless of the type of nucleic acid of the target (e.g., mRNA or cDNA). For example, in certain preferred embodiments, the profiling reagent can differentiate between the target nucleotide sequence and nucleotide sequences of other genes or (if desired) alternative nucleotide sequences of the same gene, thereby allowing the identity and quantification of the target nucleotide sequence to be determined. Typically, such a profiling reagent hybridizes to a target nucleic acid molecule by complementary base-pairing in a sequence-specific manner, and discriminates the target sequence from other nucleic acid sequences in a test sample. An example of a detection reagent is a probe that hybridizes to a target nucleic acid containing a nucleotide sequence substantially complementary to one of the sequences provided in Table 2. In a preferred embodiment, such a probe can differentiate between the target nucleic acid and nucleic acids of other genes. Another example of a detection reagent is a primer which acts as an initiation point of nucleotide extension along a complementary strand of a target polynucleotide, as in reverse transcription or PCR. The sequence information provided herein is also useful, for example, for designing primers to reverse transcribe and/or amplify (e.g., using PCR) any gene disclosed herein.

In an exemplary embodiment of the invention, a detection reagent is an isolated or synthetic DNA or RNA polynucleotide probe or primer or PNA oligomer, or a combination of DNA, RNA and/or PNA, that hybridizes to a segment of a target nucleic acid molecule corresponding to any of the genes disclosed herein. A detection reagent in the form of a polynucleotide may optionally contain modified base analogs, intercalators or minor-groove binders. Multiple detection reagents such as probes may be, for example, affixed to a solid support (e.g., arrays or beads) or supplied in solution (e.g., probe/primer sets for enzymatic reactions such as PCR, RT-PCR, TaqMan® assays, or primer-extension reactions) to form an expression profiling kit.

A probe or primer typically is a substantially purified oligonucleotide or PNA oligomer. Such oligonucleotides typically comprise a region of complementary nucleotide sequence that hybridizes under stringent conditions to at least about 8, 10, 12, 16, 18, 20, 22, 25, 30, 40, 50, 55, 60, 65, 70, 80, 90, 100, 120 (or any other number in-between) or more consecutive nucleotides in a target nucleic acid molecule.

Other preferred primer and probe sequences can readily be determined using the nucleotide sequences of genes disclosed herein. It will be apparent to one of skill in the art that such primers and probes are directly useful as reagents for expression profiling of the genes of the present invention, and can be incorporated into any kit/system format.

In order to produce a probe or primer specific for a target gene sequence, the gene/transcript sequence is typically examined using a computer algorithm which identifies oligomers of defined length that are unique to the gene sequence, have a GC content within a range suitable for hybridization, lack predicted secondary structure that may interfere with hybridization, and/or possess other desired characteristics or that lack other undesired characteristics.

A primer or probe of the present invention is typically at least about 8 nucleotides in length. In one embodiment of the invention, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, it is typically less than about 50, 60, 65, or 70 nucleotides in length, depending on the type of assay in which it is employed. In the case of a primer, it is typically less than about 30 nucleotides in length. In a specific preferred embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length.

The present invention encompasses nucleic acid analogs that contain modified, synthetic, or non-naturally occurring nucleotides or structural elements or other alternative/modified nucleic acid chemistries known in the art. Such nucleic acid analogs are useful, for example, in detection reagents (e.g., primers/probes) for detecting one or more of the genes disclosed herein. Furthermore, kits/systems (such as beads, arrays, etc.) that include these analogs are also encompassed by the present invention. For example, PNA oligomers for detecting expression of the genes disclosed herein are specifically contemplated. PNA oligomers are analogs of DNA in which the phosphate backbone is replaced with a peptide-like backbone (Lagriffoul et al., Bioorganic & Medicinal Chemistry Letters 4:1081-1082 [1994], Petersen et al., Bioorganic & Medicinal Chemistry Letters 6:793-796 [1996], Kumar et al., Organic Letters 3[9]: 1269-1272 [2001], WO96/04000). PNA hybridizes to complementary RNA or DNA with higher affinity and specificity than conventional oligonucleotides and oligonucleotide analogs. The properties of PNA enable novel molecular biology and biochemistry applications unachievable with traditional oligonucleotides and peptides.

Additional examples of nucleic acid modifications that improve the binding properties and/or stability of a nucleic acid include the use of base analogs such as inosine, intercalators (e.g., U.S. Pat. No. 4,835,263) such as ethidium bromide and SYBR® Green, and minor-groove binders (e.g., U.S. Pat. No. 5,801,115). Thus, references herein to nucleic acid molecules, expression profiling reagents (e.g., probes and primers), and oligonucleotides/polynucleotides include PNA oligomers and other nucleic acid analogs. Other examples of nucleic acid analogs and alternative/modified nucleic acid chemistries known in the art are described in Current Protocols in Nucleic Acid Chemistry, John Wiley & Sons, New York (2002).

While the design of each allele-specific primer or probe depends on variables such as the precise composition of the nucleotide sequences in a target nucleic acid molecule and the length of the primer or probe, another factor in the use of primers and probes is the stringency of the conditions under which the hybridization between the probe or primer and the target sequence is performed. Higher stringency conditions utilize buffers with lower ionic strength and/or a higher reaction temperature, and tend to require a closer match between the probe/primer and target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example but not limitation, exemplary conditions for high-stringency hybridization conditions using an allele-specific probe are as follows: prehybridization with a solution containing 5× standard saline phosphate EDTA (SSPE), 0.5% NaDodSO₄ (SDS) at 55° C., and incubating probe with target nucleic acid molecules in the same solution at the same temperature, followed by washing with a solution containing 2×SSPE, and 0.1% SDS at 55° C. or room temperature.

Moderate-stringency hybridization conditions may be used for primer extension reactions with a solution containing, e.g., about 50 mM KCl at about 46° C. Alternatively, the reaction may be carried out at an elevated temperature such as 60° C. In another embodiment, a moderately-stringent hybridization condition is suitable for oligonucleotide ligation assay (OLA) reactions, wherein two probes are ligated if they are completely complementary to the target sequence, and may utilize a solution of about 100 mM KCl at a temperature of 46° C.

In a hybridization-based assay, specific probes can be designed that hybridize to a segment of target DNA of one gene sequence but do not hybridize to sequences from other genes. Hybridization conditions should be sufficiently stringent that there is a significant detectable difference in hybridization intensity between genes, and preferably an essentially binary response, whereby a probe hybridizes to only one of the gene sequences or significantly more strongly to one gene sequence.

Oligonucleotide probes and primers may be prepared by methods well known in the art. Chemical synthetic methods include, but are not limited to, the phosphotriester method described by Narang et al., Methods in Enzymology 68:90 [1979]; the phosphodiester method described by Brown et al., Methods in Enzymology 68:109 [1979], the diethylphosphoamidate method described by Beaucage et al., Tetrahedron Letters 22:1859 [1981]; and the solid support method described in U.S. Pat. No. 4,458,066. In the case of an array, multiple probes can be immobilized on the same support for simultaneous analysis of multiple different gene sequences.

In a certain type of PCR-based assay, a gene-specific primer hybridizes to a region on a target nucleic acid molecule that overlaps a gene sequence and only primes amplification of the gene sequence to which the primer exhibits perfect complementarity (Gibbs, Nucleic Acid Res. 17:2427-2448 [1989]). Typically, the primer's 3′-most nucleotide is aligned with and complementary to a target nucleotide (e.g., a SNP). This primer is used in conjunction with a second primer that hybridizes at a distal site. Typically, amplification only proceeds if the first primer exhibits perfect complementarity (e.g., if the 3′-most nucleotide of the first primer is complementary to one of two alternative nucleotides that can be present at a SNP position that aligns with the 3′-most nucleotide of the first primer), producing a detectable product that indicates which gene/transcript variant is present in the test sample (e.g., which nucleotide is present at a target SNP site). This PCR-based assay can be utilized as part of a TaqMan® assay, for example.

The genes described herein, such as ESR1, PGR, ERBB2, NUP214, and PPIG, can be detected by any one of a variety of nucleic acid amplification methods, which are used to increase the copy numbers of a polynucleotide of interest in a nucleic acid sample. Such amplification methods are well known in the art, and they include, but are not limited to, polymerase chain reaction (PCR) (e.g., U.S. Pat. Nos. 4,683,195 and 4,683,202; PCR Technology: Principles and Applications for DNA Amplification, ed. H. A. Erlich, Freeman Press, New York, N.Y. [1992]), ligase chain reaction (LCR) (Wu and Wallace, Genomics 4:560 [1989]; Landegren et al., Science 241:1077 [1988]), strand displacement amplification (SDA) (e.g., U.S. Pat. Nos. 5,270,184 and 5,422,252), transcription-mediated amplification (TMA) (e.g., U.S. Pat. No. 5,399,491), linked linear amplification (LLA) (e.g., U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874 [1990]). Based on such methodologies, a person skilled in the art can readily design primers in any suitable regions “and” of the gene sequences of interest, so as to amplify the genes disclosed herein. Such primers may be used to reverse-transcribe and amplify nucleic acid molecules of any length, such that it contains the gene of interest in its sequence.

Generally, an amplified polynucleotide is at least about 16 nucleotides in length. More typically, an amplified polynucleotide is at least about 20 nucleotides in length. In a preferred embodiment of the invention, an amplified polynucleotide is at least about 30 nucleotides in length. In a more preferred embodiment of the invention, an amplified polynucleotide is at least about 32, 40, 45, 50, or 60 nucleotides in length. In yet another preferred embodiment of the invention, an amplified polynucleotide is at least about 100, 200, 300, 400, or 500 nucleotides in length. While the total length of an amplified polynucleotide of the invention can be as long as, for example, an exon or an entire gene, an amplified product is typically up to about 1,000 nucleotides in length (although certain amplification methods may generate amplified products greater than 1,000 nucleotides in length). In certain embodiments, an amplified polynucleotide is not greater than about 150-250 nucleotides in length.

In an embodiment of the invention, a gene expression profiling reagent of the invention is labeled with a fluorogenic reporter dye that emits a detectable signal. While the preferred reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment of the invention, the detection reagent may be further labeled with a quencher dye such as Tamra, especially when the reagent is used as a self-quenching probe such as in a TaqMan assay (e.g., U.S. Pat. Nos. 5,210,015 and 5,538,848) or Molecular Beacon probe (e.g., U.S. Pat. Nos. 5,118,801 and 5,312,728), or other stemless or linear beacon probe (Livak et al., PCR Method Appl. 4:357-362 [1995]; Tyagi et al., Nature Biotechnology 14:303-308 [1996]; Nazarenko et al., Nucl. Acids Res. 25:2516-2521 [1997]; U.S. Pat. Nos. 5,866,336 and 6,117,635).

The detection reagents of the invention may also contain other labels, including but not limited to, biotin for streptavidin binding, hapten for antibody binding, and oligonucleotide for binding to another complementary oligonucleotide such as pairs of zipcodes.

Gene Expression Kits and Systems

A person skilled in the art will recognize that, based on the gene and sequence information disclosed herein, expression profiling reagents can be developed and used to assay any genes of the present invention individually or in combination, and such detection reagents can be readily incorporated into one of the established kit or system formats which are well known in the art. The terms “kits” and “systems,” as used herein in the context of gene expression profiling reagents, are intended to refer to such things as combinations of multiple gene expression profiling reagents, or one or more gene expression profiling reagents in combination with one or more other types of elements or components (e.g., other types of biochemical reagents, containers, packages such as packaging intended for commercial sale, substrates to which gene expression profiling reagents are attached, electronic hardware components, etc.). Accordingly, the present invention further provides gene expression profiling kits and systems, including but not limited to, packaged probe and primer sets (e.g., TaqMan® probe/primer sets), arrays/microarrays of nucleic acid molecules, and beads that contain one or more probes, primers, or other detection reagents for profiling one or more genes of the present invention. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more gene expression profiling reagents (along with, optionally, other biochemical reagents) packaged in one or more containers.

In some embodiments, a gene expression profiling kit typically contains one or more detection reagents and other components (e.g., a buffer; enzymes such as reverse transcriptase, DNA polymerases, or ligases; reverse transcription and chain extension nucleotides such as deoxynucleotide triphosphates; in the case of Sanger-type DNA sequencing reactions, chain terminating nucleotides; positive control sequences; negative control sequences; and the like) necessary to carry out an assay or reaction, such as reverse transcription, amplification, and/or detection of a nucleic acid molecule. A kit may further contain means for determining the amount of a target nucleic acid, and means for comparing the amount with a standard, and can comprise instructions for using the kit to detect the nucleic acid molecule of interest. In certain embodiments of the invention, kits are provided which contain the necessary reagents to carry out one or more assays to profile the expression of one or more of the genes disclosed herein. In certain embodiments of the invention, gene expression profiling kits/systems are in the form of nucleic acid arrays, or compartmentalized kits, including microfluidic/lab-on-a-chip systems.

Gene expression profiling kits/systems may contain, for example, one or more probes, or pairs of probes, that hybridize to a nucleic acid molecule at or near each target gene sequence position. Multiple pairs of gene-specific probes may be included in the kit/system to simultaneously assay a plurality of genes, at least one of which is a gene of the present invention. In some kits/systems, the gene-specific probes are immobilized to a substrate such as an array or bead. For example, the same substrate can comprise gene-specific probes for detecting at any or all of ESR1, PGR, ERBB2, NUP214, and PPIG, particularly both ESR1 and PGR optionally also in combination with ERBB2.

The terms “arrays,” “microarrays” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In certain embodiments, the microarray is prepared and used according to the methods described in U.S. Pat. No. 5,837,832 (Chee et al.), PCT application WO95/11995 (Chee et al.), Lockhart, D. J. et al. (Nat. Biotech. 14:1675-1680 [1996]) and Schena, M. et al. (Proc. Natl. Acad. Sci. 93:10614-10619 [1996]), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics,” Biotechnol. Annu. Rev. 8:85-101 (2002); Sosnowski et al., “Active microelectronic array system for DNA hybridization, genotyping and pharmacogenomic applications,” Psychiatr. Genet. 12(4):181-92 (December 2002); Heller, “DNA microarray technology: devices, systems, and applications,” Annu. Rev. Biomed. Eng. 4:129-53 (2002); Epub Mar. 22 2002; Kolchinsky et al., “Analysis of SNPs and other genomic variations using gel-based chips,” Hum. Mutat. 19(4):343-60 (April 2002); and McGall et al., “High-density genechip oligonucleotide probe arrays,” Adv. Biochem. Eng. Biotechnol. 77:21-42 (2002).

Any number of probes, such as gene-specific probes, may be implemented in an array, and each probe or pair of probes can hybridize to a different gene sequence position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. The microarray or detection kit can contain polynucleotides that cover the known 5′ or 3′ sequence of a gene/transcript, sequential polynucleotides that cover the full-length sequence of a gene/transcript; or unique polynucleotides selected from particular areas along the length of a target gene/transcript sequence. Polynucleotides used in the microarray or detection kit can be specific to a gene or genes of interest (e.g., specific to a particular signature sequence within a target gene sequence, or specific to a particular gene sequence at multiple different sequence sites), or specific to a polymorphic gene/transcript or genes/transcripts of interest. Hybridization assays based on polynucleotide arrays rely on the differences in hybridization stability of the probes to perfectly matched and mismatched target sequences.

In certain embodiments, the arrays are used in conjunction with chemiluminescent detection technology. The following patents and patent applications, which are all herein incorporated by reference in their entirety, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,938, 5,843,681, 5,800,999, and 5,773,628 describe methods and compositions of dioxetane for performing chemiluminescent detection; and U.S. published application US2002/0110828 discloses methods and compositions for microarray controls.

In certain embodiments of the invention, a nucleic acid array can comprise an array of probes of about 15-25 nucleotides in length. In further embodiments, a nucleic acid array can comprise any number of probes, in which at least one probe is capable of detecting one or more genes selected from the group consisting of ESR1, PGR, ERBB2, NUP214, and PPIG (particularly ESR1, PGR, and optionally ERBB2), and/or at least one probe comprises a fragment of one of the gene sequences disclosed herein, and sequences complementary thereto, said fragment comprising at least about 8 consecutive nucleotides, preferably 10, 12, 15, 16, 18, 20, more preferably 22, 25, 30, 40, 47, 50, 55, 60, 65, 70, 80, 90, 100, or more consecutive nucleotides (or any other number in-between) and containing (or being complementary to) a sequence of a gene selected from the group consisting of ESR1, PGR, ERBB2, NUP214, and PPIG (particularly ESR1, PGR, and optionally ERBB2).

A polynucleotide probe can be synthesized on the surface of a substrate by using a chemical coupling procedure and an ink jet application apparatus, such as described in PCT application WO95/251116 (Baldeschweiler et al.), which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, exemplary embodiments of the invention provide methods of identifying and profiling expression of the genes disclosed herein in a test sample. Such methods typically involve incubating a test sample of nucleic acids with an array comprising one or more probes corresponding to at least one gene sequence of the invention, and assaying for binding of a nucleic acid from the test sample with one or more of the probes. Conditions for incubating a gene expression profiling reagent (or a kit/system that employs one or more such gene expression profiling reagents) with a test sample vary. Incubation conditions depend on factors such as the format employed in the assay, the profiling methods employed, and the type and nature of the profiling reagents used in the assay. One skilled in the art will recognize that any one of the commonly available hybridization, amplification and array assay formats can readily be adapted to detect the genes disclosed herein.

A gene expression profiling kit/system of the present invention may include components that are used to prepare nucleic acids from a test sample for the subsequent reverse transcription, RNA enrichment, amplification and/or detection of a nucleic acid molecule. Such sample preparation components can be used to produce nucleic acid extracts (including DNA, cDNA and/or RNA) from any tumor tissue source, including but not limited to, fresh tumor biopsy, frozen, or FFPE tissue specimens, or tumors collected and preserved by any method. The test samples used in the above-described methods will vary based on such factors as the assay format, nature of the profiling method, and the specific tissues, cells, or extracts used as the test sample to be assayed. Methods of preparing nucleic acids are well known in the art and can be readily adapted to obtain a sample that is compatible with the system utilized. Automated sample preparation systems for extracting nucleic acids from a test sample are commercially available, and examples include Qiagen's BioRobot 9600 and QIAcube, Thermo Scientific Kingfisher® Purification Systems, and Roche Molecular Systems' COBAS AmpliPrep System.

Another form of kit contemplated by the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other gene expression profiling reagent for profiling the expression of one or more genes of the present invention, one or more containers which contain wash reagents (such as phosphate buffered saline, Tris-buffers, etc.), and one or more containers which contain the reagents used to reveal the presence of the bound probe or other gene expression profiling reagents. The kit can optionally further comprise compartments and/or reagents for, for example, reverse transcription, RNA enrichment, nucleic acid amplification, or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art (see, e.g., Weigl et al., “Lab-on-a-chip for drug development,” Adv. Drug Deliv. Rev. 24, 55(3):349-77 (February 2003)). In such microfluidic devices, the containers may be referred to as, for example, microfluidic “compartments,” “chambers,” or “channels.”

The gene expression profiling reagents of the invention, such as the nucleic acid molecules provided in Table 2, have a variety of uses, especially in the determination of ER, PR, and/or ERBB2 status, such as for the diagnosis, prognosis, or treatment of breast cancer (e.g., selection of a therapeutic agent). For example, the nucleic acid molecules are useful as amplification primers or hybridization probes, such as for expression profiling of messenger RNA, transcript RNA, cDNA, genomic DNA, amplified DNA or other nucleic acid molecules, and for isolating full-length cDNA and genomic clones encoding the genes disclosed herein (e.g., the ESR1, PGR, and ERBB2 genes, as well as the housekeeping genes NUP214 and PPIG).

Thus, the nucleic acid molecules of the invention can be used as, for example, reverse transcription and/or amplification primers and hybridization probes to detect and profile the expression levels of the genes disclosed herein, particularly for breast cancer assessment.

Calculation of mRNA Expression Levels and Gene Status

In certain exemplary embodiments, expression levels of the genes disclosed herein (e.g., ESR1, PGR, and/or ERBB2) may be calculated by the Δ(ΔCₜ) method (interchangeably referred to as the ΔΔC_{T} method; see Livak et al., Methods 2001, 25:402-408), where Ct=the threshold cycle for target amplification; i.e., the cycle number in PCR at which time exponentional amplification of target begins. (K J Livak and T D Schmittgen, 2001, Methods 25:402-408). The level of mRNA of each of the profiled genes may be defined as:

Δ(ΔCt)=(−1)×(Ct_{GOI}−Ct_{EC})_{test RNA}−(Ct_{GOI}−Ct_{EC})_{ref RNA}

where GOI=gene of interest (e.g., ESR1, PGR, and/or ERBB2), test RNA=RNA obtained from the patient sample, ref RNA=a calibrator reference RNA, and EC=an endogenous control (e.g., NUP214 and/or PPIG). The expression level of each gene to be detected (e.g., ESR1, PGR, and/or ERBB2) may be first normalized to one or more endogenous control genes, such as the two housekeeping genes NUP214 and PPIG. A Ct representing the average of the Cts obtained from amplification of the two endogenous controls (Ct_{EC}) can be used to minimize the risk of normalization bias that may occur if only one control gene were used (T. Suzuki, P J Higgins et al., 2000, Biotechniques 29:332-337). Exemplary primers that may be used to amplify the endogenous control genes are listed in Table 2 (but primers for amplifying these endogenous control genes are not limited to these disclosed oligonucleotides). The adjusted expression level of the gene(s) of interest may be further normalized to a calibrator reference RNA pool, such as ref RNA (Universal Human Reference RNA, Stratagene, La Jolla, Calif.), or other control sample. This can be used to standardize expression results obtained from various machines.

The Δ(ΔCₜ) method (which is interchangeably referred to as ΔΔC_{T}) is described in, for example, Livak et al., Methods 2001, 25:402-408. ΔΔC_{T} values calculated from ESR1, PGR, and ERBB2 expression levels can be applied to classify the expression levels of these genes as “positive” or “negative” with respect to ER, PR, and ERBB2 status, respectively. For example, ΔΔC_{T} cutoff points can be selected and used to classify ΔΔC_{T} values for ESR1, PGR, and ERBB2 expression levels that are above (or equal to) the cutoff as “positive” with respect to ER, PR, and ERBB2 status (respectively), and/or to classify ΔΔC_{T} values for ESR1, PGR, and ERBB2 expression levels that are below (or equal to) the cutoff as “negative” with respect to ER, PR, and ERBB2 status (respectively). Alternatively, various clustering methods based on ΔΔC_{T} can be employed for the same purposes. Clustering methods are described in, for example, Fraley et al., J Am Stat Assoc 2002, 97:611-631, Fraley et al., J Class 1999, 16:297-306, and Ma et al., J Clin Oncol 2006, 24: 4611-4619.

A wide variety of statistical methods and thresholds can be used for determining or classifying ER, PR, and/or ERBB2 status (as well as the status of other hormonal receptors and/or growth factor receptors) from mRNA expression levels of these genes. See Dudoit et al., “Classification in Microarray Experiments”, Statistical Analysis of Gene Expression Microarray Data, 2003, Chapman & Hall/CRC: 93-158, incorporated herein by reference in its entirety, for examples of methods known in the art for classifying gene expression data.

For example, with respect to threshold levels, a wide variety of cut-offs can be employed for classifying the status of a gene, such as classifying ER, PR, and/or ERBB2 status as positive or negative. Methods for selecting or formulating these cut-offs are known in the art and/or can be implemented by one of ordinary skill in the art. For classifying the expression status of a given gene, various discrete “cutoffs” or continuous classification systems can be applied. For example, the classification of ER, PR, and/or ERBB2 status as positive or negative can be accomplished using a variety of methods. Certain methods may involve using a set of training data to produce a model that can then be used to classify the status of test samples. For example, positive/negative cutoffs can be selected by manual inspection of a training data set, and these cutoffs can be applied to classifying test samples. As an example, a test sample in which expression of a given gene (e.g., ESR1, PGR, or ERBB2), which may be indicated by ΔΔC_{T} or other statistical methods, is above (or equal to) a pre-determined cutoff can be classified as “positive” whereas a test sample in which expression of the gene is below (or equal to) the pre-determined cutoff can be classified as “negative”. Thus, the cutoff can be used as a benchmark when compared to the expression level of a given gene (e.g., ESR1, PGR, or ERBB2) in a breast cancer patient, such as to classify the status of that gene (e.g., as “positive” or “negative” with respect to ER, PR, or ERBB2 status). This status can then be used, for example, by a medical practitioner to formulate or select a treatment strategy or therapeutic agent best suited for the breast cancer patient.

“Example One” (below) describes exemplary statistical methods for classifying ER, PR, and ERBB2 status based on either ΔΔC_{T} cutoffs or clustering methods. However, these statistical methods, as well as the thresholds (e.g., cutoffs) employed for classifying gene status, are merely exemplary, and one of ordinary skill in the art will appreciate that many alternative statistical methods, classification systems, and thresholds can be employed, particularly to determine ER, PR, and/or ERBB2 status from the mRNA expression levels of these genes. In Example One, the results of mRNA expression analysis of breast cancer specimens were used as training data to develop two classification methods, a cutoff point method (cutoffs were selected based on IHC Allred scores) and a clustering method (which classified ER or PR status independent of IHC Allred scores), which were then validated in further sample sets. In Example One, the ΔΔC_{T} values of ER, PR, and ERBB2 in various breast tumor samples were calculated. Using these ΔΔC_{T} values in the cutoff point method, ER, PR, and ERBB2 status were classified using ΔΔC_{T} cutoff points of 1.5 for ER, 0.5 for PR, and 3.5 for ERBB2, and the receptor status was classified as positive if ΔΔC_{T} was greater than or equal to the cutoff point. Using these ΔΔC_{T} values in the clustering method to classify ER and PR status, a Gaussian mixture model as implemented in MCLUST software was employed to define clusters of subjects based on ER ΔΔC_{T} and PR ΔΔC_{T} measurements. The mixture models estimated from the training data were then used to assign test subjects to the cluster for which they had the highest probability of membership based on their ΔΔC_{T} measurements.

The ΔΔC_{T} cutoff points of 1.5 for ER, 0.5 for PR, and 3.5 for ERBB2 used in Example One below are merely exemplary cutoff points, and other cutoff points can also be used. Examples of alternative ΔΔC_{T} cutoff points for ER include, but are not limited to, any values between about 1 and 2, inclusive. Examples of alternative ΔΔC_{T} cutoff points for PR include, but are not limited to, any values between about 0 and 1, inclusive. Examples of alternative ΔΔC_{T} cutoff points for ERBB2 include, but are not limited to, any values between about 3 and 4, inclusive.

Clustering Methods

As an alternative to the ΔΔC_{T} cutoff-point method, clustering methods can also be used for classifying samples (such as to classify hormonal receptor and/or growth factor receptor status such as ER, PR, and/or HER2 status, or the status of any other gene(s) of interest).

As an example, parameters for ER, PR, and HER2 can be derived from discovery sample sets (see “Example One” below) using Gaussian mixture modeling implemented in MCLUST (“R: A Language and Environment for Statistical Computing”, R Development Core Team, R Foundation for Statistical Computing; Banfield et al., Biometrics 1993, 49:803-821; Fraley et al., J Class 1999, 16:297-306; Fraley et al., Technical Report No. 415, Dept. of Statistics, Univ. of Washington, October 2002; Fraley et al., J Am Stat Assoc 2002, 97:611-631; and Fraley et al., J Class 2003, 20:263-286). Exemplary parameters derived for ER, PR, and HER2 are listed in Table 21. Parameters such as the exemplary parameters listed in Table 21 can be used with π value and ΔΔC_{T} to calculate probability and confidence for classifying a sample, such as in the following example (using ER as an example):

1) Calculate probability:

$$p {y}_{ER -} = \frac{{e}^{\frac{- {( ΔΔ {C}_{T} - {U}_{ER -} )}^{2}}{2 \times {V}_{ER -}}}}{\sqrt{2 π \times {V}_{ER -}}}$$

$$p {y}_{ER +} = \frac{{e}^{\frac{- {( ΔΔ {C}_{T} - {U}_{ER +} )}^{2}}{2 \times {V}_{ER +}}}}{\sqrt{2 π \times {V}_{ER +}}}$$

π=3.1415926535 and e=2.71828182845905

2) Determine confidence:

$${Z}_{ER -} = \frac{p {y}_{ER -} \times {p}_{ER -}}{( p {y}_{ER -} \times {p}_{ER -} ) + ( p {y}_{ER +} \times {p}_{ER +} )}$$

$${Z}_{ER +} = \frac{p {y}_{ER +} \times {p}_{ER +}}{( p {y}_{ER -} \times {p}_{ER -} ) + ( p {y}_{ER +} \times {p}_{ER +} )}$$

3) Classification of status:
ER+=z_{ER+}≧z_{ER-}
ER−=z_{ER+}<z_{ER-}
Uncertainty of ER+=1−Z_{ER+}
Uncertainty of ER−=1−Z_{ER-}

Absolute Quantitation Methods

In addition to relative quantitation methods, absolute quantitation methods can also be used for classifying samples (such as to classify hormonal receptor and/or growth factor receptor status such as ER, PR, and/or HER2 status, or the status of any other gene(s) of interest).

Absolute quantitation methods can optionally be done without using a control sample (such as for monitoring experiment-to-experiment variation).

In absolute quantitation methods, the expression level of ER, PR, and HER2 (or other gene(s) of interest) in a sample can optionally be normalized with the expression level of one or more control genes (such as either or both of the housekeeping genes NUP214 and PPIG), such as follows:

ΔC_{T sample}=C_{T} of gene of interest−C_{T} of HSK genes

Using (−1)×ΔC_{T sample} data from ER, PR, HER2 discovery sample sets, exemplary ΔC_{T} cutoff values for absolute quantitation were defined as −3.4, −5.1, and −1.0 for ER, PR, and HER2, respectively. Examples of alternative ΔC_{T} cutoff points for absolute quantitation of ER include, but are not limited to, any values between about −3.9 and −2.9, inclusive. Examples of alternative ΔC_{T} cutoff points for absolute quantitation of PR include, but are not limited to, any values between about −5.6 and −4.6, inclusive. Examples of alternative ΔC_{T} cutoff points for absolute quantitation of HER2 include, but are not limited to, any values between about −1.5 and −0.5, inclusive.

### EXAMPLES

The following examples are offered to illustrate, but not to limit, the claimed invention.

### Example One

### A Single-Tube Quantitative Assay for mRNA Levels of Hormonal and Growth Factor Receptors in Breast Cancer Specimens (Multiplex Taqman® Assay for ER, PR & HER2)

Overview

A single-tube, one-step multiplex TaqMan® reverse transcription-polymerase chain reaction (RT-PCR) assay was developed to quantitate mRNA levels of ER, PR, HER2, and two housekeeping genes (referred to herein as the “mERPR+HER2” assay) in breast cancer FFPE sections. Using data from discovery sample sets, IHC-status-dependent cutoff-point and IHC-status-independent clustering methods for classification of receptor status were evaluated, and then were validated with independent sample sets. When compared to IHC status, the accuracies of the mERPR+HER2 assay with the cutoff-point classification method were 0.98 (95% CI: 0.97-1.00), 0.92 (95% CI: 0.88-0.95), and 0.97 (95% CI: 0.95-0.99) for ER, PR, and HER2, respectively, for the validation sets. Furthermore, the areas under the receiver operating characteristic (ROC) curves were 0.997 (95% CI: 0.994-1.000), 0.967 (95% CI: 0.949-0.985), and 0.968 (95% CI: 0.915-1.000) for ER, PR, and HER2, respectively. This multiplex assay provides a sensitive and reliable method to quantitate hormonal and growth factor receptors.

See Iverson et al., “A Single-Tube Quantitative Assay for mRNA Levels of Hormonal and Growth Factor Receptors in Breast Cancer Specimens”, (J Mol Diagn. 11 (2) 2009 (in press)), incorporated herein by reference in its entirety (including the “Supplemental Materials”; FIGS. 1-6, S1A, S1B, S2A, and S2B; and “Figure Legends” section).

Materials and Methods

Study Subjects

Three sets of formalin-fixed, paraffin-embedded (FFPE) breast tumor sections were used to develop the RT-PCR assay for ER, PR and HER2. Two contemporary sets (“sample set 1” and “sample set 2”) were provided by Laboratory Corporation of America® (LabCorp®), and a third set of archived FFPE breast tumor samples (“sample set 3”) was provided by Guy's and St Thomas' Tissue and Data bank (London, United Kingdom). The cohort of 291 subjects was diagnosed between 1975 and 2001 with tumor size <3 cm, lymph node negative and ER-positive (ER+) primary breast tumors, and the use of this cohort was approved by Guy's Research Ethics Committee (04/Q0704/137). The use of these three sample sets for the development of classification methods of hormonal and growth factor receptors, and the number of samples with IHC Allred scores for each sample set are listed in Table 1.

Immunohistochemistry (IHC) Assays

Hormonal Receptors

For the IHC assay performed at LabCorp®, the FFPE tissue specimens were mounted on SuperFrost Plus slides (Fisher Scientific, Hampton, N.H.) and dried for 30 minutes in a 60° C. slide drier. A hematoxylin and eosin (H&E) stained section was prepared for each specimen and evaluated for the presence of tumor cells. The FFPE slides were processed on the BenchMark XT Autostainer (Ventana Medical Systems, Tucson, Ariz.). The primary monoclonal antibodies used to detect ER and PR were anti-estrogen receptor clone 6F11 and anti-progesterone clone 16 (Ventana Medical Systems), respectively. The sequence of primary staining events on the automated stainer included: incubations with primary antibodies; application of a biotinylated secondary antibody; binding of avidin-biotin-horseradish peroxidase complex; and detection with diaminobenzidine (DAB) chromagen. After staining, the slides were counterstained and evaluated by a pathologist for hormone receptor status, which involved evaluation of at least 200 tumor cells to determine the percentage of stained cells as well as the intensity of staining.

Guy's and St Thomas' Tissue and Data Bank specimens were collected between 1975 and 2001, therefore the hormonal receptor status was re-evaluated with contemporary IHC assays. Each FFPE block was cut in the following sequence: one section for H&E staining, six unstained sections on charged slides for IHC, a second section for H&E staining followed by five 10 μm sections on charged glass slides. All section cutting was carried out in RNase-free conditions. On the second H&E stained slide, areas with tumor were marked on the cover slip and this guide slide was sent with the 10-μm sections to facilitate macro-dissection of tumor areas for RNA extraction. In order to standardize ER or PR status assessment, all cases were re-evaluated. The anti-estrogen receptor a antibody (SP-1) and anti-progesterone receptor (PgR636) were used in a conventional IHC protocol for ER and PR status, respectively. Briefly, sections were pre-treated by pressure cooking in citrate buffer pH6 prior to incubating with SP-1 or PgR636. Sites of antigen-antibody binding were detected using the Dako REAL Envision™ system. This set of specimens was also used for the discovery of a prognostic signature for distant metastasis; therefore ER, PR, and HER2 status were re-evaluated independently by two pathologists (Tutt et al., “Risk estimation of distant metastasis in node-negative, estrogen receptor-positive breast cancer patients using an RT-PCR based prognostic expression signature”, BMC Cancer (in press)). Any discrepant scores were then assessed jointly and a final score agreed upon.

Allred scores based on the percentage of tumor cells (PS), intensity of the staining (IS), and total score (TS=PS+IS) were recorded for all three sets of specimens (Allred et al., Mod Pathol 1998, 11:155-168). The distributions of Allred PS, IS, and TS for both ER and PR in the three sample sets are listed in Tables 9-11.

Growth Factor Receptor HER2

HercepTest™ reagents (Dako, Carpinteria, Calif.) with Dako Autostainer and with Biogenex i6000 autostainer (San Ramon, Calif.) were used for sample set 2 and sample set 3, respectively. Sample set 2 was scored according to the criteria with cell membrane staining indicated as 3+(strong, complete membrane staining in >10% of tumor cells), 2+(weak to moderate, complete membrane staining in >10% of the tumor cells), 1+(faint membrane staining that involves only a portion of the membrane, in >10% of tumor cells) or 0 (no staining observed, or faint staining in <10% of the tumor cells). For sample set 3, HER2 IHC was scored according to the new ASCO-CAP guidelines (Wolff et al., Arch Pathol Lab Med 2007, 131:18-43).

RNA Extraction from FFPE Sections

All FFPE section slides used for this study were 4- or 10-μm thick with ˜60 to 80% breast tumor cells. The FFPE section slides were deparaffinized by soaking them in xylene for 10 minutes with occasional agitation and repeated with fresh xylene. The slides were then washed consecutively with 100% ethanol, 90% ethanol, and 70% ethanol with 2 minutes for each wash. The slides were then air dried at room temperature for 5 minutes. Fifteen microliters of Proteinase K digestion solution [2 mg/mL Proteinase K (Ambion, Austin, Tex.), 0.1 M NaCl, 10 mM Tris pH 8.0, 1 mM EDTA, and 0.5% SDS], was applied to the dried tissue on the slide. The tissue was then scraped with a sterile surgical blade and transferred into a 1.5 mL tube containing 185 μL Proteinase K digestion solution, and incubated overnight at 55° C. for 18 to 24 hours. After incubation, the samples were spun at 14,000 rpm for 5 minutes, and the supernatant was transferred to a new tube. A mixture of 600 μL of 100% ethanol and 400 μL of extraction buffer (5 M Guanidium thiocyanate, 31.25 mM Na Citrate, pH 7.0, 0.625% Sarcosyl, and 0.125 M β-mercaptoethanol) was added to the supernatant of each sample, loaded into Zymo-Spin II Columns (Zymo Research, Orange, Calif.), spun at 12,000 rpm for one minute, and repeated until the entire sample had been spun through the column. The column was washed once with 200 μL of wash buffer (80% ethanol in 10 mM Tris-HCl and 0.1 mM EDTA, pH 8.0), followed by 13.5 Kunitz units DNase (QIAGEN, Valencia, Calif.) treatment at room temperature for 30 minutes. The columns were washed with 200 μL wash buffer twice and then dried by centrifugation for 2 minutes at 12,000 rpm. The total RNA was then eluted twice with 50 μL of TE buffer that had been heated to 65° C.

The amount of PCR-amplifiable RNA was quantitated by one-step RT-PCR using primers for the housekeeping (HSK) gene, NUP214, and compared to a serially diluted control, Universal Human Reference RNA (Stratagene, La Jolla, Calif.). The recovery of amplifiable RNA depends on the age of the FFPE specimen and RNA extraction methods. The recovery of amplifiable RNA from one 4-μm breast cancer FFPE section ranges from 0.5 ng to 25 ng.

A New Approach for Determining Normalization Factor

The top two most stable HSK genes, PPIG and NUP214, were previously determined by the profiling of 138 breast cancer FFPE samples (Tutt et al., “Risk estimation of distant metastasis in node-negative, estrogen receptor-positive breast cancer patients using an RT-PCR based prognostic expression signature”, BMC Cancer (in press)), and they were used to validate the novel approach of determining the normalization factor for RNA amount in each RT-PCR reaction. Fifty-eight human total RNA samples (see Table 12) from various tissue types were used to demonstrate the feasibility of using two TaqMan® probes labeled with identical fluorescent reporter dye (see Table 13) to determine the normalization factor of total RNA input amount in each sample. The concentration of each RNA sample was determined using RiboGreen® quantitation assay (Invitrogen, Carlsbad, Calif.), and 20 ng of total RNA was used for each reaction. The expression levels of two HSK genes, NUP214 or PPIG, were quantitated in independent simplex reactions using either NUP214 probe or PPIG probe labeled with the same fluorescent reporter dye using the 7900 Real-Time PCR System (“7900 system”) (Applied Biosystems, Foster City, Calif.). The average of NUP214 and PPIG expression levels was then compared to the composite NUP214 and PPIG expression level quantitated using both NUP214 and PPIG TaqMan® probes in a single reaction.

Single-Tube, One-Step Multiplex TaqMan® Assays

mERPR+HER2 RT-PCR Assay on the 7500 System

Table 2 lists gene IDs, gene symbols, the oligonucleotide sequences of PCR primers, the accession numbers of RefSeq and GenBank in National Center for Biotechnology Information (NCBI) of known splice variants amplified by the designed PCR primers for ESR1, PGR, ERBB2 (HER2), and the two HSK genes, NUP214 and PPIG, and the oligonucleotide sequences and fluorescent reporters of all TaqMan® probes for the 7500 Real-Time PCR System (“7500 system”) (Applied Biosystems, Foster City, Calif.).

Quantitative detection of mRNA levels of ESR1, PGR, ERBB2 (HER2), and two HSK genes in a single tube was accomplished through one-step five-plex TaqMan® RT-PCR assay. Each reaction contained 50 mM of Tricine, 115 mM KOAc (pH 8.0), 4.5 mM Mn(OAc)₂, 7.4% glycerol, 400 μM dATP, 400 μM dGTP, 400 μM dCTP, 800 μM dUTP, 1% DMSO, 50 nM NPR (provided by Applied Biosystems) in 5% Tween-20, 0.12 μM enhancer (Abbott, Abbott Park, Ill.), 0.08 unit/μL Uracil N-glycosylase, 0.4 unit/μL Z05 DNA polymerase (Abbott, Abbott Park, Ill.), 500 nM of each primer (Applied Biosystems, Foster City, Calif.), 250 nM of each TaqMan® probe (Applied Biosystems, Foster City, Calif.), and approximately 0.2 to 1 ng of amplifiable RNA extracted from the FFPE specimen. TRE and PHO labeled TaqMan® probes were provided by Applied Biosystems, (U.S. Pat. Nos. 6,080,852, 5,847,162, 6,025,505, and 6,017,712). The thermocycling parameters were as follows: 50° C. for 2 minutes; 95° C. for 1 minute; 60° C. for 30 minutes; 95° C. for 15 seconds and 58° C. for 35 seconds for 42 cycles for the 7500 system. In addition to each RNA sample from the FFPE specimen, 25 ng of the Universal Human Reference RNA was included as the control in each amplification plate, and all samples were run in duplicate reactions.

mERPR RT-PCR Assay on the 7900 System

A single-tube multiplex TaqMan® assay for ER, PR, and two HSKs (“mERPR” assay) was developed for the 7900 system. The mERPR+HER2 assay for the 7900 system was not developed due to the unavailability of a compatible florescent dye for HER2 for the optical system on the 7900 system. Table 13 lists the oligonucleotide sequences, orientations, fluorescent reporters, and quenchers of all TaqMan® probes for the 7900 system.

Quantitative detection of mRNA levels of ER, PR, and two housekeeping genes in a single tube was also accomplished through one-step multiplex TaqMan® RT-PCR with a 384-well plate using the 7900 system. Each 15 μL reaction contained 50 mM of Tricine, 115 mM KOAc (pH 8.0), 4.5 mM Mn(OAc)₂, 9.6% glycerol, 400 μM dATP, 400 μM dGTP, 400 μM dCTP, 800 μM dUTP, 1% DMSO, 0.3 μM 6-ROX (Invitrogen, Carlsbad, Calif.) in 5% Tween-20, 0.12 μM enhancer (Abbott, Abbott Park, Ill.), 0.08 unit/μL Uracil N-glycosylase, 0.4 unit/μL Z05 DNA polymerase (Abbott, Abbott Park, Ill.), 500 nM of each primer, 200 nM TET-labeled (or NED-labeled) TaqMan® probes for each HSK gene, 250 nM FAM-labeled TaqMan® probe for ER, 250 nM VIC-labeled TaqMan® probe for PR, and approximately 0.5 to 1 ng of amplifiable RNA extracted from FFPE specimens. The thermocycling parameters for the 7900 system are as follows: 50° C. for 2 minutes; 95° C. for 1 minute; 60° C. for 30 minutes; 95° C. for 15 seconds and 58° C. for 30 seconds for 42 cycles. In addition to each RNA sample from FFPE specimens, 25 ng of the Universal Human Reference RNA (Stratagene, La Jolla, Calif.) was included as the control in each amplification plate. All samples on the plate were run in duplicate.

FFPE Section-to-Section Reproducibility

To determine FFPE section-to-section reproducibility, five sequential sections from each of 10 breast cancer tumor FFPE samples (BioChain Institute, Hayward, Calif.) were obtained. Before RNA was isolated, the slide was checked to ensure that all sections from each sample were identical in size and shape. Total RNA was extracted from these 50 sections and the recovery was determined using NanoDrop (Thermo Scientific, Wilmington, Del.). The amplifiable RNA was determined by a TaqMan® RT-PCR assay for the housekeeping gene, NUP214. ER, PR and HER2 mRNA levels in each section were determined using the mERPR+HER2 assay.

Data Analysis

The ER, PR, and HER2 mRNA expression levels in each FFPE clinical sample were calculated using the ΔΔC_{T} method (Livak et al., Methods 2001, 25:402-408). First, the average C_{T} (cycle threshold) of duplicate reactions of each gene of interest was calculated for each sample and the control sample, Universal Human Reference RNA. Then the ER, PR, and HER2 mRNA expression levels were normalized with the HSK gene expression level for each FFPE and the control sample. Finally, the HSK-normalized ER, PR, and HER2 expression levels in each FFPE sample were further compared to the HSK-normalized ER, PR, and HER2 expression levels in the control sample, respectively. Therefore, the relative expression level of each gene of interest in each FFPE sample, is presented as ΔΔC_{T}=(−1)×[ΔC_{T sample} (C_{T} of gene of interest−C_{T} of HSK genes)−ΔC_{T control} (C_{T} of gene of interest−C_{T} of HSK genes)]. A minus one factor is included to graphically illustrate higher expression above lower expression. When C_{T} value was not reported, then a C_{T} of 42 was used for the calculation of ΔΔC_{T}.

Statistical Analysis

For ER and PR classification, the results of the mERPR+HER2 assay from sample set 1 and combined sample sets 2 and 3 were used as the discovery and validation sets, respectively. For HER2 classification, the results of the mERPR+HER2 assay from sample set 2 and sample set 3 were used as the discovery and validation sets, respectively.

Area under the receiver operating characteristic curve (AUC) measures the ability of the assay to discriminate between positive and negative status of ER, PR, or normal- and over-expression status of HER2 across the entire range of ΔΔC_{T} values. AUC was computed based on the ROC function available from the Mayo Clinic, and confidence intervals (CI) for the AUC were calculated using the variance estimate described by Delong et al. (Biometrics 1988, 44:837-845).

Two different methods were used to classify the status of ER, PR, and HER2. An IHC-status-dependent ΔΔC_{T} cutoff-point method was used to determine the hormonal and growth factor receptor status. Using IHC status as the gold standard, an Allred score ≧3 defines positive hormonal status (ER+ or PR+) (Allred et al., Mod Pathol 1998, 11:155-168), and an intensity score of HER2 3+ defines HER2 overexpression (Wolff et al., Arch Pathol Lab Med 2007, 131:18-43). The ΔΔC_{T} cutoff point for classification of each marker was empirically selected based on the diagnostic metrics of sensitivity, specificity, positive predictive value (PPV), negative predictive value (NPV), and accuracy from the comparisons with IHC status using various ΔΔC_{T} cutoff points. A ΔΔC_{T} cutoff point for classification of each marker was selected using the data from their respective discovery sets. The selected ΔΔC_{T} cutoff points were then applied to classify ER, PR and HER2 status of samples in their respective validation sets.

An IHC-status-independent classification method was established by developing Gaussian mixture models as implemented in MCLUST software for the R programming language (“R: A Language and Environment for Statistical Computing”, R Development Core Team, R Foundation for Statistical Computing) based solely on ER ΔΔC_{T}, PR ΔΔC_{T}, and HER2 ΔΔC_{T} measurements of subjects in their respective discovery sets (Banfield et al., Biometrics 1993, 49:803-821; Fraley et al., J Class 1999, 16:297-306; Fraley et al., Technical Report No. 415, Dept. of Statistics, Univ. of Washington, October 2002; Fraley et al., J Am Stat Assoc 2002, 97:611-631; Fraley et al., J Class 2003, 20:263-286). The Bayesian Information Criterion (BIC) was used to determine the best fitting model. For ER and HER2 measures, the best model was a mixture of two Gaussian distributions with equal variance. For PR, since the best model by Bayesian Information Criterion was a single Gaussian distribution which would not be helpful for classification purposes, a mixture model of two Gaussian distributions with equal variance was used. The mixture models estimated from the discovery data were then used to classify an independent set of validation subjects to the cluster for which they had the highest probability of membership based on their ΔΔC_{T} measurements.

The diagnostic metrics of sensitivity, specificity, PPV, NPV, and accuracy were calculated for both discovery and validation sets. The agreement coefficient, Cohen's kappa (Cohen et al., Educ Psychol Meas 1960, 20:37-46), was used to evaluate the agreement between the IHC status and the status determined using the results from the mERPR+HER2 assay for the ΔΔC_{T} cutoff-point and clustering methods. In addition, the square of Pearson's correlation coefficient was used to assess the degree of correlation between two instrument platforms.

Results

A New Approach for Determination of Normalization Factor

In order to obtain more accurate normalization of RNA input amount and to accommodate three genes of interest, ESR1, PGR, and ERBB2, in a multiplex TaqMan® assay with four different fluorescent reporters, a novel approach of determining the expression levels of two HSK genes using two TaqMan® probes labeled with the same fluorescent reporter was designed.

Two HSK genes, NUP214 and PPIG, expressed at relatively constant levels in breast tumor FFPE specimens were selected to validate the approach. mRNA levels of NUP214 and PPIG were averaged from independent reactions with NUP214 or PPIG probes, and compared with the NUP214 and PPIG composite mRNA level in a single co-amplification reaction. 58 total RNA samples from various tissues were compared using the two amplification formats. The two different formats of determining HSK gene expression levels correlated well, with a correlation coefficient, r², of 0.9742 (p<0.0001).

FFPE Section-to-Section Reproducibility

Total RNA and amplifiable RNA from each of five sequential sections of 10 breast cancer tumor FFPE samples were determined by absorbance at 260 nm and the TaqMan® RT-PCR assay for the housekeeping gene NUP214. The average amplifiable RNA from 10 FFPE samples varied from 70 ng (S4) to 1300 ng (S1). Relatively larger variations of the PR ΔΔC_{T} values in samples S2, S4, and S8 were due to later C_{T} resulting from lower PGR expression levels. There was no correlation between the variation of amplifiable RNA recovery and ER, PR, or HER2 ΔΔC_{T} values.

Classification of Hormonal Receptor Status

Three breast cancer tumor FFPE sample sets with available ER and PR IHC Allred total scores listed in Table 1 were used to determine the classifications of ER and PR status. Sample set 1 (with 67 samples) and combined sample sets 2 and 3 (with 333 samples) were used as the discovery and validation sets, respectively. Both ER mRNA and PR mRNA were detected in all clinical specimens using the mERPR+HER2 assay.

Estrogen Receptor

The ER ΔΔC_{T} values of 67 RNA samples of the discovery set using the mERPR+HER2 assay were calculated, and the distribution of ER ΔΔC_{T} values in the discovery set was bimodal as reported previously (Ma et al., J Clin Oncol 2006, 24:4611-4619). The AUC of ER ΔΔC_{T} values from the discovery set was 0.989 (95% CI: 0.972-1.000). The performance measurements of sensitivity, specificity, PPV, NPV, and accuracy for the ER classification based on the IHC ER status were compared using various ΔΔC_{T} cutoff points (cutoff points were 0, 0.5, 1, 1.5, 2, 2.5, 3, 3.5, 4, 4.5, and 5). A ΔΔC_{T} cutoff point of 1.5 with 94% accuracy was empirically selected to divide 67 ER ΔΔC_{T} values into two groups. The distribution of 67 IHC ER Allred total scores and the classifications of ER status by both the IHC-status-dependent ΔΔC_{T} cutoff-point and the IHC-status-independent clustering methods are listed in Table 3. Two Allred TS0 samples and two Allred TS3 samples were classified as ER+ and ER−, respectively, by the ΔΔC_{T} cutoff-point method. All Allred TS0 samples were classified as ER− correctly, and two Allred TS3 samples were classified as ER− by the clustering method. When compared to IHC ER status, the kappa coefficient of the clustering method, 0.924 (95% CI: 0.821-1.000) was higher than that of the ΔΔC_{T} cutoff-point method, 0.842 (95% CI: 0.693-0.992) (Table 4).

Both the ΔΔC_{T} cutoff point of 1.5 and the model parameters for the clustering method derived from the discovery set were applied to classify the ER status of samples in the validation set. The validation set consisted of two independent subsets, sample set 2 and sample set 3 (listed in Table 1). Forty-two samples with ER IHC Allred scores in sample set 2 and 291 samples with ER IHC Allred scores in sample set 3 were used to validate ER classification. The 291 archived specimens in sample set 3 were originally identified as ER+ between 1975 and 2001. The ER and PR status was re-evaluated in these specimens with contemporary IHC assays, and 8 of 291 samples (3%) were re-classified as IHC ER−. The AUC of ER ΔΔC_{T} values from the validation set was 0.997 (95% CI: 0.994-1.000). The distribution of IHC Allred total scores of the entire 333 samples and the classifications of ER status by both the IHC-status-dependent ΔΔC_{T} cutoff-point and the IHC-status-independent clustering methods of the validation set are listed in Table 3. One Allred TS0 sample and four Allred TS3 samples were classified as ER+ and ER−, respectively, by the ΔΔC_{T} cutoff-point method. All IHC ER− samples were correctly classified as ER− by the clustering method. However, an additional six Allred TS4 to TS6 samples and one Allred TS8 sample were classified as ER− by the clustering method. When compared to IHC ER status, the kappa coefficient of the clustering method was 0.759 (95% CI: 0.623-0.895), lower than the 0.870 (95% CI: 0.758-0.982) of the ΔΔC_{T} cutoff-point method (Table 4).

Progesterone Receptor

The performance measurements of the PR classification of 67 ΔΔC_{T} values based on the IHC PR status were compared using various ΔΔC_{T} cutoff points (cutoff points were −1.5, −1, −0.5, 0, 0.5, 1, 1.5, 2, 2.5, 3, and 3.5). The AUC of PR ΔΔC_{T} values from the discovery set was 0.987 (95% CI: 0.969-1.000). A ΔΔC_{T} cutoff point of 0.5 with 94% accuracy was empirically selected to divide 67 PR ΔΔC_{T} values into two groups. The distribution of 67 IHC PR Allred total scores and the classifications of PR status by both the IHC-status-dependent ΔΔC_{T} cutoff-point and the IHC-status-independent clustering methods are listed in Table 5. One Allred TS0 sample was classified as PR+ by both the ΔΔC_{T} cutoff-point and clustering methods. One Allred TS3 and two Allred TS5 samples were classified as PR− by the ΔΔC_{T} cutoff-point method, and three additional samples (one Allred TS4, one Allred TS5, and one Allred TS6) were also classified as PR− by the clustering method. When compared to IHC PR status, the kappa coefficients of the ΔΔC_{T} cutoff-point and clustering methods were 0.861 (95% CI: 0.730-0.993) and 0.767 (95% CI: 0.607-0.928), respectively (Table 6).

Both the ΔΔC_{T} cutoff point of 0.5 and the model parameters for the clustering method derived from the discovery set were applied to classify PR status of samples in the validation set. The validation set also consisted of two independent subsets, sample set 2 and sample set 3 (listed in Table 1). Forty-two samples with PR IHC Allred scores and 279 samples with PR IHC Allred scores from sample set 2 and sample set 3, respectively, were used to validate PR classification. The AUC of PR ΔΔC_{T} values from the validation set was 0.967 (95% CI: 0.949-0.985). The distribution of IHC Allred total scores and the classifications of PR status of 321 validation samples by both the ΔΔC_{T} cutoff-point and the IHC-status-independent clustering methods are listed in Table 5. Twelve samples (11 Allred TS0 and one Allred TS2) and eight samples (seven Allred TS0 and one Allred TS2) were classified as PR+ by the ΔΔC_{T} cutoff-point method and the clustering method, respectively. Fourteen Allred TS3 and TS4 samples were classified as PR− by the ΔΔC_{T} cutoff-point method, and an additional six samples (four Allred TS3, one Allred TS5, and one Allred TS6) were classified as PR− by the clustering method. When compared to IHC PR status, the kappa coefficients of the ΔΔC_{T} cutoff-point and clustering methods were similar but lower than those of the discovery set, 0.664 (95% CI: 0.544-0.784) and 0.669 (95% CI: 0.556-0.782), respectively (Table 6).

Classification of Overexpression of Growth Factor Receptor HER2

The HER2 ΔΔC_{T} values of 55 samples of the HER2 discovery set (sample set 2 in Table 1) using the mERPR+HER2 assay were determined. The AUC of HER2 ΔΔC_{T} values from the discovery set was 0.968 (95% CI: 0.924-1.000). The HER2 ΔΔC_{T} values were compared to HER2 IHC scores with HER2 IHC 3+ (HER2-over) defined as samples expressing above the normal level of HER2 (HER2-norm). The performance measurements of HER2 classification based on the HER2 IHC status were compared using various HER2 ΔΔC_{T} cutoff points (cutoff points were 1.5, 2, 2.5, 3, 3.5, 4, 4.5, 5, 5.5, and 6). A ΔΔC_{T} cutoff point of 3.5 with 91% accuracy was empirically selected to divide 55 HER2 ΔΔC_{T} values into two groups. The distribution of HER2 IHC scores and the classification of HER2 status by both ΔΔC_{T} cutoff-point and clustering methods of the discovery set are listed in Table 7. Using a ΔΔC_{T} cutoff point of 3.5 for the classification of HER2 expression status, one HER2 IHC 2+ sample was classified as HER2-over, and four samples with HER2 IHC 3+ were classified as HER2-norm. Using the clustering method, all 38 samples with HER2 IHC 0 to 2+ were classified correctly. Nine of 17 samples with HER2 IHC 3+ were classified as HER2-norm. When compared to IHC HER2 expression status, the kappa coefficients of the ΔΔC_{T} cutoff-point and clustering methods for classification of HER2 expression status of the discovery set were 0.776 (95% CI: 0.592-0.961) and 0.551 (95% CI: 0.312-0.791), respectively (Table 8).

Both the ΔΔC_{T} cutoff point of 3.5 and the model parameters for the clustering method derived from the discovery set were applied to classify HER2 expression status of 272 samples in the validation set. The AUC of HER2 ΔΔC_{T} values from the validation set was 0.968 (95% CI: 0.915-1.000). The distribution of 272 HER2 IHC scores and the classification of HER2 status by both ΔΔC_{T} cutoff-point and clustering methods of the validation set are listed in Table 7. Using the ΔΔC_{T} cutoff point of 3.5, four samples (two HER2 IHC 0 and two HER2 IHC 1+) were classified as HER2-over, and three HER2 IHC 3+ samples were classified as HER2-norm. Using the clustering method, all 255 HER2-norm samples were classified correctly, but 12 of 17 HER2 IHC 3+ samples were classified as HER2-norm. When compared to IHC HER2 expression status, the kappa coefficients of the ΔΔC_{T} cutoff-point and clustering methods for classification of HER2 overexpression of the validation set were 0.786 (95% CI: 0.633-0.940) and 0.439 (95% CI: 0.182-0.696), respectively (Table 8).

Diagnostic Metrics of mERPR+HER2 Assay

The performance measurements of the mERPR+HER2 assay, sensitivity, specificity, positive predictive value (PPV), negative predictive value (NPV), accuracy, and kappa coefficient, for ER, PR, and HER2 overexpression with the discovery and validation sets are listed in Tables 4, 6, and 8, respectively.

All ΔΔC_{T} values from the discovery and validation sets were sorted, and then plotted using ΔΔC_{T} of 1.5, 0.5, and 3.5 as the cutoff points for ER, PR, and HER2, respectively, and compared with IHC ER, PR, and HER2 status.

Discussion

A multiplex TaqMan® assay to quantitate mRNA levels of ER, PR, HER2, and two HSK genes in a single tube was developed. A multiplex assay in a single tube for these genes is particularly useful in that small amounts of RNA may be recovered from FFPE sections (Esteva et al., Clin Cancer Res 2005, 11:3315-3319 and Chang et al., Breast Cancer Res Treat 2008, 108:233-240). This may be due to such factors as the size of the tissue biopsy, the type of the fixative, the age of the paraffin block, or the degree of chemical modification, any of which may affect the recovery of amplifiable RNA from FFPE sections. The performance of the mERPR+HER2 assay, which is especially useful for breast cancer diagnosis, was evaluated with three sets of breast cancer specimens using two classification methods on two instrument platforms.

The results of the evaluation of breast cancer FFPE sections using the mERPR+HER2 assay demonstrated good reproducibility for samples with ER+, PR+, or HER2-over status, and better than that of the group of ER−, PR−, or HER2-norm, respectively, because of the later C_{T} values resulting from the relatively low abundance of mRNA levels.

The lack of intermediate Allred scores in the ER discovery sample set (only two Allred TS3 and no Allred TS2 or TS4 samples) rendered the ΔΔC_{T} cutoff-point selection more challenging; therefore the more conservative lower ΔΔC_{T} cutoff point of 1.5 was selected. Approximately two thirds of breast cancer has ER+ status, however sample set 3 of the validation sample set in this study was mostly ER+ (97%). Consequently, the percentage of samples with HER2 overexpression (HER2 IHC 3+) in this set was also lower than the generally observed 25% to 30% with HER2 overexpression (Arpino et al., J Natl Cancer Inst 2005, 97:1254-1261). The kappa coefficients of ER classification using the ΔΔC_{T} cutoff-point method for the discovery and validation sets were similar, 0.842 and 0.870, respectively (Table 4). In contrast, the kappa coefficient of ER classification using the clustering method dropped from 0.924 to 0.759 for the validation set (Table 4). The discordant results between the IHC ER assay and the mERPR+HER2 assay were nine (2%) and 13 (3%) of a total of 400 samples using the ΔΔC_{T} cutoff-point method and the clustering method, respectively.

The ER mRNA expression in breast tumor specimens is bimodal as represented by the sigmoidal transition between RT-PCR− and RT-PCR+ groups. Both IHC ER−/PCR ER+ and IHC ER+/PCR ER− groups were identified by IHC methods with different antibodies used by the two clinical sites. Therefore, it is likely that the performance of the different antibodies was similar even though the SP1 clone used by Guy's Hospital has been indicated to have higher affinity and a more robust performance (Gown et al., Mod Pathol 2008, 21:S8-S15 and Cheang et al., J Clin Oncol 2006, 24:5637-5644). IHC ER− but PCR ER+ subjects, which are not being identified by IHC, may merit consideration for endocrine therapy.

The kappa coefficients of the agreement of ER status between the IHC assay and the mERPR+HER2 assay with the ΔΔC_{T} cutoff-point method was “almost perfect” (Landis et al., Biometrics. 1977, 33:159-174) based on the interpretation of Cohen's kappa for both discovery and validation sets, thus supporting the cutoff point of 1.5 (36 out of 400 samples in the discovery and validation sets were IHC ER−). The agreement of ER status between the IHC assay and the mERPR+HER2 assay with the ΔΔC_{T} cutoff-point method was slightly higher than those reported by Cronin et al. (Am J Pathol 2004, 164:35-42) (kappa=0.825; n=62) and Ma et al. (J Clin Oncol 2006, 24:4611-4619) (kappa=0.83; n=852). Subsequently, two additional groups reported the agreement of ER status between the IHC assay and the ER TaqMan® assay in the Oncotype DX™ as kappa=0.81 (n=149) (Esteva et al. Clin Cancer Res 2005, 11:3315-3319) and kappa=1.0 (n=80) (Chang et al., Breast Cancer Res Treat 2008, 108:233-240).

As compared to ER mRNA expression, PR mRNA expression is generally more continuous as represented by a gradual increase of ΔΔC_{T} values from the RT-PCR− group to the RT-PCR+ group. The kappa coefficients of PR status between the IHC assay and the mERPR+HER2 assay dropped from the discovery to validation set using both ΔΔC_{T} cutoff-point and the clustering methods (Table 6). When compared to ER discordant results, the percentage of samples with discordant results between PR IHC assay and the mERPR+HER2 assay were larger, 30 (8%) and 25 (6%) of a total of 388 samples using ΔΔC_{T} cutoff-point method and the clustering method, respectively, which is likely due to the more continuous values for expression of PR. The agreement of PR status between the IHC assay and the mERPR+HER2 assay with the ΔΔC_{T} cutoff-point method for the validation set was similar to those reported by Cronin et al. (Am J Pathol 2004, 164:35-42) (kappa=0.674; n=62) and Ma et al. (J Clin Oncol 2006, 24:4611-4619) (kappa=0.70; n=852). However, subsequently two groups reported lower agreement for PR status, kappa of 0.48 (n=149) (Esteva et al., Clin Cancer Res 2005, 11:3315-3319) and kappa of 0.57 (n=80), using the PR TaqMan® assay in the Oncotype DX™ (Chang et al., Breast Cancer Res Treat 2008, 108:233-240).

The performances of ER and PR classifications using IHC-status-dependent ΔΔC_{T} cutoff-point and IHC-status-independent clustering methods were similar (Tables 4 and 6). The performance of classification of HER2 overexpression between the IHC-status-dependent ΔΔC_{T} cutoff-point and IHC-status-independent clustering methods differed (Table 8). Using the clustering method, 9 of 17 samples (53%) and 12 of 17 samples (70%) with HER2 IHC 3+ samples were classified as HER2-norm for the discovery and validation sets, respectively. Based on the clustering results, a HER2 ΔΔC_{T} cutoff point of 5.0 instead of 3.5 could have been selected to classify HER2 status, which would have a sensitivity of HER2 classification in the discovery set of 0.47 compared to the IHC assay. The agreement, kappa, of HER2 overexpression status between the IHC assay and the mERPR+HER2 assay with the ΔΔC_{T} cutoff-point method for both discovery and validation sets (Table 8) were higher than kappa of 0.60 with the HER2 TaqMan® assay in the Oncotype DX™ (Esteva et al., Clin Cancer Res 2005, 11:3315-3319).

The exemplary embodiment of the invention described in this example is a sensitive single-tube, one-step multiplex TaqMan® assay to quantitate ER, PR, and HER2 expression levels. Results from this assay were consistent across multiple adjacent sections from the same breast tumor. The classification of ER, PR, and HER2-overexpression status was evaluated with two methods and compared with IHC results. Based on the interpretation of kappa coefficients, the agreement was “almost perfect” for ER, and the agreement was “substantial” for both PR and HER2 (Landis et al., Biometrics. 1977, 33:159-174). This RT-PCR assay to determine the ER, PR, and HER2 status can be used, for example, in a clinical laboratory for molecular testing of predictive and prognostic markers for breast cancer. Furthermore, determining quantitative ER, PR, and HER2 expression levels may also be useful for determining resistance to tamoxifen and non-responsiveness to trastuzmab treatments.

### Example Two

### Using Multiplex TaqMan® Assays to Profile a Prognostic Signature for Breast Cancer

Overview

In order to reduce the required RNA amount recovered from formalin-fixed, paraffin-embedded sections (FFPE) and decrease the number of assays for a multi-gene assay, five multiplex TaqMan® assays were developed to profile a previously reported SYBR® Green-based 14-gene prognostic signature for breast cancer (which is described in U.S. patent application Ser. No. 12/012,530, Kit Lau et al., filed Jan. 31, 2008, incorporated herein by reference in its entirety). The performance of the multiplex TaqMan® assays was validated in clinical samples.

Methods

Five multiplex RT-PCR TaqMan® assays were designed to quantitatively measure the mRNA levels of a prognostic signature which comprised 14 genes of interest and 3 housekeeping (HSK) genes. The 14 genes of interest were as follows: CENPA, PKMYT1, MELK, MYBL2, BUB1, RACGAP1, TK1, UBE2S, C16orf61 (DC13), RFC4, PRR11(FLJ11029), DIAPH3, ORC6L, and CCNB1. The 3 HSKs were PPIG, NUP214, and SLU7. These 14 genes of interest and 3 HSKs are described in U.S. patent application Ser. No. 12/012,530, Kit Lau et al., filed Jan. 31, 2008, which is incorporated herein by reference in its entirety, and are also shown in Table 19 of the instant application (Table 19 of the instant application corresponds with Table 2 of U.S. patent application Ser. No. 12/012,530). In addition, assays to quantitate mRNA levels of hormonal receptors, ESR1 and PGR, and growth factor receptor, ERBB2, were also included. Twenty genes were divided into five 4-plex assays with 4 fluorescent reporters in each multiplex. Total RNA was extracted from FFPE sections of 35 breast cancer patient samples from Guy's Hospital in the United Kingdom. The gene expression levels were quantified using the 7500 Real-time PCR System (Applied Biosystems). A control sample, Universal Human Reference RNA (Stratagene), was included in each run. The ΔΔC_{T} (the difference between HSK genes normalized C_{T} of the sample and HSK genes normalized C_{T} of the control) for each of 14 genes were first calculated for each sample, and then the sum of all 14 ΔΔC_{T} (SDD) of each sample and two predetermined cutoffs were used to determine three categories of prognostic risk (low, moderate, and high). The SDD results and risk calls from multiplex TaqMan® assays and simplex SYBR® Green assays were compared.

Results

The five 4-plex TaqMan® assays were first evaluated with RNA from five commonly used breast cancer cell lines. There was a significant correlation between the SYBR® Green assay and multiplex TaqMan® assays. The correlation coefficient, R², for SDD was 0.984. The status of ESR1, PGR, and ERBB2 genes of 5 cell lines were consistent with those reported in the literature. For 35 clinical specimens, the correlation coefficient, R², was 0.977. 31 of 35 (89%) risk category calls were identical to those determined by SYBR® Green assays. Discordance mainly occurred in the intermediate category. The correlation coefficient, R², between SYBR® Green and multiplex TaqMan assays for ESR1 and PGR were 0.991 and 0.915, respectively.

Thus, five 4-plex TaqMan® assays were developed to profile a 14-gene prognostic signature plus the hormonal receptors ESR1 and PGR and growth factor receptor ERBB2 for breast cancer. These TaqMan® assays can be used for the quantitative measurement of mRNA levels in specimens with low RNA yield, for example, and facilitate high throughput testing.

All publications and patents cited in this specification are herein incorporated by reference in their entirety. Various modifications and variations of the described compositions, methods and systems of the invention will be apparent to those skilled in the art without departing from the scope and spirit of the invention. Although the invention has been described in connection with specific preferred embodiments and certain working examples, it should be understood that the invention as claimed should not be unduly limited to such specific embodiments. Indeed, various modifications of the above-described modes for carrying out the invention that are obvious to those skilled in the field of molecular biology, genetics and related fields are intended to be within the scope of the following claims.

TABLE 1
Description of sample sets used for data analyses

| Sample Set | Subject No. | Discovery | Validation |
|---|---|---|---|
| Set 1* | 67 | ER, PR | |
| Set 2 | 55 | HER2 | ER, PR^{†} |
| Set 3 | 291 | | ER, PR, HER2^{‡} |

*HER2 IHC status was not available,
^{†}ER and PR IHC Allred scores were available for 42 of 55 samples,
^{‡}ER, PR, and HER2 IHC Allred scores were available for 291, 279, and 272 of 291 samples, respectively. A total of 400, 388, and 327 samples with ER, PR, and HER2 IHC status, respectively, were used for data analyses.

TABLE 2
Genes and information of exemplary RT-PCR primers and
TaqMan ® probes in the mERPR+HER2 assay

| Gene | Gene | Accession | Forward Primer | Reversed Primer | | Probe Sequence |
|---|---|---|---|---|---|---|
| ID | Symbol | Number | Sequence (5′→3′) | Sequence (5′→3′) | Reporter | (5′→3′)^{§} |
| 2099 | ESR1* | NM_000125 | TCTGCAGGGAGAGGAGTTT | GGTCCTTCTCTTCCAGAGACTT | 6FAM | TGTGCCTCAAATCTA (SEQ |
| | | (SEQ ID NO:16) | (SEQ ID NO:1) | (SEQ ID NO:2) | | ID NO:3) |
| 5241 | PGR* | NM_000926 | TCGAGTCATTACCTCAGAAGAT | CCCACAGGTAAGGACACCATA | TRE^{‡} | TGACAGCCTGATGCTTCAT |
| | | (SEQ ID NO:20) | (SEQ ID NO:4) | (SEQ ID NO:5) | | (SEQ ID NO:6) |
| 2064 | ERBB2^{†} | NM_004448 | CAGCCCTGGTCACCTACAA | GGGACAGGCAGTCACACA | PHO^{‡} | TGAGTCCATGCCCAATCC |
| | | (SEQ ID NO:24) | (SEQ ID NO:7) | (SEQ ID NO:8) | | (SEQ ID NO:9) |
| | | NM_001005862 | | | | |
| | | (SEQ ID NO:25) | | | | |
| 8021 | NUP214 | NM_005085 | CATTTGCTTTATAAAAGACCACTG | CCACTCCAAGTCTAGAACATCA | VIC | TCAGGAAATTCGGCGCCTT |
| | | (SEQ ID NO:26) | (SEQ ID NO:10) | (SEQ ID NO:11) | | (SEQ ID NO:12) |
| 9360 | PPIG | NM_004792 | GCCAACAGAGGGAAGGATA | GAGGAGTTGGTTTCGTTGTTA | VIC | ATGGTTCACAGTTCTTC |
| | | (SEQ ID NO:27) | (SEQ ID NO:13) | (SEQ ID NO:14) | | (SEQ ID NO:15) |

*ESR1 and PGR have at least four alternative splice variants. AF258449 (SEQ ID NO:17), AF258450 (SEQ ID NO:18), and AF258451 (SEQ ID NO:19) are the accession numbers of three other variants for ESR1. AB085683 (SEQ ID NO:21), AB085844 (SEQ ID NO:22), and AB085845 (SEQ ID NO:23) are the accession numbers of three other variants for PGR.
^{†}ERBB2 was annotated with two splice variants. For each of these genes, RT-PCR primers were designed to amplify a region shared by all listed splice variants. The amplicon sizes using these primers are 104-bp, 80-bp, 95-bp, 123-bp, and 61-bp, for ESR1, PGR, ERBB2, NUP214, and PPIG, respectively.
^{‡}TRE and PHO labeled probes were provided by Applied Biosystems.
^{§}TaqMan ® probes can have minor-groove binder and non-fluorescent quencher at 3′ termini.
Alternative primers for NUP214:
forward (5′→3′): ACTGGATCCCAAGAGTGAAG (SEQ ID NO:28)
reversed (5′→3′): TCACATCTTGGACAGCAAAT (SEQ ID NO:29)

TABLE 3
Classification of ER status of the discovery and validation sets
using the ΔΔC_{T} cutoff-point and clustering methods.

| | Discovery (n = 67) | | | | | Validation (n = 333) | | | | |
|---|---|---|---|---|---|---|---|---|---|---|
| Allred | IHC | ΔΔC_{T} cutoff-point | | Clustering | | IHC | ΔΔC_{T} cutoff-point | | Clustering | |
| TS* | (% of total) | ER+ | ER− | ER+ | ER− | (% of total) | ER+ | ER− | ER+ | ER− |
| 0 | 17 | 2 | 15 | 0 | 17 | 17 | 1 | 16 | 0 | 17 |
| 2 | 0 | 0 | 0 | 0 | 0 | 2 | 0 | 2 | 0 | 2 |
| ER−^{†} | 17 (25%) | 2 | 15 | 0 | 17 | 19 (6%) | 1 | 18 | 0 | 19 |
| 3 | 2 | 0 | 2 | 0 | 2 | 4 | 0 | 4 | 0 | 4 |
| 4 | 0 | 0 | 0 | 0 | 0 | 2 | 2 | 0 | 1 | 1 |
| 5 | 3 | 3 | 0 | 3 | 0 | 6 | 6 | 0 | 4 | 2 |
| 6 | 2 | 2 | 0 | 2 | 0 | 31 | 31 | 0 | 28 | 3 |
| 7 | 12 | 12 | 0 | 12 | 0 | 110 | 110 | 0 | 110 | 0 |
| 8 | 31 | 31 | 0 | 31 | 0 | 161 | 161 | 0 | 160 | 1 |
| ER+^{‡} | 50 (75%) | 48 | 2 | 48 | 2 | 314 (94%) | 310 | 4 | 303 | 11 |

*Allred total score.
^{†}Total number of specimens with Allred TS0 and TS2 in each set.
^{‡}Total number of specimens with Allred TS3 to TS8 in each set.

TABLE 4
Summary of the performance of ER classification

| | Discovery (n = 67) | | Validation (n = 333) | |
|---|---|---|---|---|
| | ΔΔC_{T} cutoff-point | Clustering | ΔΔC_{T} cutoff-point | Clustering |
| Sensitivity | 0.96 (0.86-1.00) | 0.96 (0.86-1.00) | 0.99 (0.97-1.00) | 0.96 (0.94-0.98) |
| Specificity | 0.88 (0.64-0.99) | 1.00 (0.80-1.00) | 0.95 (0.74-1.00) | 1.00 (0.82-1.00) |
| PPV | 0.96 (0.86-1.00) | 1.00 (0.93-1.00) | 1.00 (0.98-1.00) | 1.00 (0.99-1.00) |
| NPV | 0.88 (0.64-0.99) | 0.89 (0.67-0.99) | 0.82 (0.60-0.95) | 0.63 (0.44-0.80) |
| Accuracy | 0.94 (0.85-0.98) | 0.97 (0.90-1.00) | 0.98 (0.97-1.00) | 0.97 (0.94-0.98) |
| Kappa | 0.842 (0.693-0.992) | 0.924 (0.821-1.000) | 0.870 (0.758-0.982) | 0.759 (0.623-0.895) |

TABLE 5
Classification of PR status of the discovery and validation sets
using the ΔΔC_{T} cutoff-point and clustering methods

| | Discovery (n = 67) | | | | | Validation (n = 321) | | | | |
|---|---|---|---|---|---|---|---|---|---|---|
| Allred | IHC | ΔΔC_{T} cutoff-point | | Clustering | | IHC | ΔΔC_{T} cutoff-point | | Clustering | |
| TS* | (% of total) | PR+ | PR− | PR+ | PR− | (% of total) | PR+ | PR− | PR+ | PR− |
| 0 | 20 | 1 | 19 | 1 | 19 | 35 | 11 | 24 | 7 | 28 |
| 2 | 0 | 0 | 0 | 0 | 0 | 9 | 1 | 8 | 1 | 8 |
| PR−^{†} | 20 (30%) | 1 | 19 | 1 | 19 | 44 (14%) | 12 | 32 | 8 | 36 |
| 3 | 1 | 0 | 1 | 0 | 1 | 36 | 24 | 12 | 20 | 16 |
| 4 | 3 | 3 | 0 | 2 | 1 | 28 | 26 | 2 | 26 | 2 |
| 5 | 11 | 9 | 2 | 8 | 3 | 51 | 51 | 0 | 50 | 1 |
| 6 | 11 | 11 | 0 | 10 | 1 | 47 | 47 | 0 | 46 | 1 |
| 7 | 7 | 7 | 0 | 7 | 0 | 58 | 59 | 0 | 58 | 0 |
| 8 | 14 | 14 | 0 | 14 | 0 | 57 | 56 | 0 | 57 | 0 |
| PR+^{‡} | 47 (70%) | 44 | 3 | 41 | 6 | 277 (86%) | 263 | 14 | 257 | 20 |

*Allred total score.
^{†}Total number of specimens with Allred TS0 and TS2 in each set.
^{‡}Total number of specimens with Allred TS3 to TS8 in each set.

TABLE 6
Summary of the performance of PR classification

| | Discovery (n = 67) | | Validation (n = 321) | |
|---|---|---|---|---|
| | ΔΔC_{T} cutoff-point | Clustering | ΔΔC_{T} cutoff-point | Clustering |
| Sensitivity | 0.94 (0.82-0.99) | 0.87 (0.74-0.95) | 0.95 (0.92-0.97) | 0.93 (0.89-0.96) |
| Specificity | 0.95 (0.75-1.00) | 0.95 (0.75-1.00) | 0.73 (0.57-0.85) | 0.82 (0.67-0.92) |
| PPV | 0.98 (0.88-1.00) | 0.98 (0.87-1.00) | 0.96 (0.93-0.98) | 0.97 (0.94-0.99) |
| NPV | 0.86 (0.65-0.97) | 0.76 (0.55-0.91) | 0.70 (0.54-0.82) | 0.64 (0.50-0.77) |
| Accuracy | 0.94 (0.85-0.98) | 0.90 (0.80-0.96) | 0.92 (0.88-0.95) | 0.91 (0.88-0.94) |
| Kappa | 0.861 (0.730-0.993) | 0.767 (0.607-0.928) | 0.664 (0.544-0.784) | 0.669 (0.556-0.782) |

TABLE 7
Classification of HER2 overexpression of the discovery and validation
sets using the ΔΔC_{T} cutoff-point and clustering methods

| | Discovery (n = 55) | | | | | Validation (n = 272) | | | | |
|---|---|---|---|---|---|---|---|---|---|---|
| | | ΔΔC_{T} cutoff-point | | Clustering | | | ΔΔC_{T} cutoff-point | | Clustering | |
| HER2 IHC | IHC | HER2- | HER2- | HER2- | HER2- | IHC | HER2- | HER2- | HER2- | HER2- |
| Score | (% of total) | over | norm | over | norm | (% of total) | over | norm | over | norm |
| 0 | 10 | 0 | 10 | 0 | 10 | 200 | 2 | 198 | 0 | 200 |
| 1+ | 20 | 0 | 20 | 0 | 20 | 53 | 2 | 51 | 0 | 53 |
| 2+ | 8 | 1 | 7 | 0 | 8 | 2 | 0 | 2 | 0 | 2 |
| HER2-norm* | 38 (69%) | 1 | 37 | 0 | 38 | 255 (94%) | 4 | 251 | 0 | 255 |
| 3+ | 17 | 13 | 4 | 8 | 9 | 17 | 14 | 3 | 5 | 12 |
| HER2-over^{†} | 17 (31%) | 13 | 4 | 8 | 9 | 17 (6%) | 14 | 3 | 5 | 12 |

*Total number of specimens with HER2 IHC scores 0, 1+, and 2+.
^{†}The number of specimens with HER2 IHC score 3+.

TABLE 8
Summary of the performance of HER2 classification

| | Discovery (n = 55) | | Validation (n = 272) | |
|---|---|---|---|---|
| | ΔΔC_{T} cutoff-point | Clustering | ΔΔC_{T} cutoff-point | Clustering |
| Sensitivity | 0.76 (0.50-0.93) | 0.53 (0.28-0.77) | 0.82 (0.57-0.96) | 0.71 (0.44-0.90) |
| Specificity | 0.97 (0.86-1.00) | 1.00 (0.91-1.00) | 0.98 (0.96-0.99) | 1.00 (0.99-1.00) |
| PPV | 0.93 (0.66-1.00) | 1.00 (0.63-1.00) | 0.78 (0.52-0.94) | 1.00 (0.48-1.00) |
| NPV | 0.90 (0.77-0.97) | 0.81 (0.67-0.91) | 0.99 (0.97-1.00) | 0.96 (0.92-0.98) |
| Accuracy | 0.91 (0.80-0.97) | 0.84 (0.71-0.92) | 0.97 (0.95-0.99) | 0.96 (0.92-0.98) |
| Kappa | 0.776 (0.592-0.961) | 0.551 (0.312-0.791) | 0.786 (0.633-0.940) | 0.439 (0.182-0.696) |

TABLE 9
Distributions of immunohistochemistry (IHC) Allred proportion
score (PS), intensity score (IS), and total score (TS) for ER
and PR of sample set 1 (for both the 7500 and 7900 systems)

| Allred | ER (n = 67) | | | PR (n = 67) | | |
|---|---|---|---|---|---|---|
| TS | No. | Allred PS* | Allred IS* | No. | Allred PS* | Allred IS* |
| 0 | 17 | 0 (17) | 0 (17) | 20 | 0 (20) | 0 (20) |
| 2 | 0 | 0 (0) | 0 (0) | 0 | 0 (0) | 0 (0) |
| HR−^{†} | 17 (25%) | | | 20 (30%) | | |
| 3 | 2 | 2 (2) | 1 (2) | 1 | 2 (1) | 1 (1) |
| 4 | 0 | 0 (0) | 0 (0) | 3 | 2 (3) | 2 (3) |
| 5 | 3 | 2 (1), 3 (2) | 2 (2), 3 (1) | 11 | 2 (9), 3 (2) | 2 (2), 3 (9) |
| 6 | 2 | 3 (1), 4 (1) | 2 (1), 3 (1) | 11 | 3 (11) | 3 (11) |
| 7 | 12 | 4 (1), 5 (11) | 2 (11), 3 (1) | 7 | 4 (6), 5 (1) | 2 (1), 3 (6) |
| 8 | 31 | 5 (31) | 3 (31) | 14 | 5 (14) | 3 (14) |

| | | |
|---|---|---|
| HR+^{‡} | 50 (75%) | 47 (70%) |

*The number of specimens is listed in the parenthesis after the Allred PS or IS.
^{†}Total number of hormone receptor negative (HR−) specimens.
^{‡}Total number of hormone receptor positive (HR+) specimens.

TABLE 10
Distributions of IHC Allred PS, IS, and TS for ER
and PR of sample set 2 (for the 7500 system only)

| Allred | ER (n = 42) | | | PR (n = 42) | | |
|---|---|---|---|---|---|---|
| TS | No. | Allred PS* | Allred IS* | No. | Allred PS* | Allred IS* |
| 0 | 11 | 0 (11) | 0 (11) | 18 | 0 (18) | 0 (18) |
| 2 | 0 | 0 (0) | 0 (0) | 0 | 0 (0) | 0 (0) |
| HR−^{†} | 11 (26%) | | | 18 (43%) | | |
| 3 | 3 | 2 (3) | 1 (3) | 1 | 2 (1) | 1 (1) |
| 4 | 0 | 0 (0) | 0 (0) | 2 | 3 (2) | 1 (2) |
| 5 | 0 | 0 (0) | 0 (0) | 2 | 4 (1), 3 (1) | 1 (1), 2 (1) |
| 6 | 3 | 5 (1), 4 (2) | 1 (1), 2 (2) | 3 | 4 (2), 3 (1) | 2 (2), 3 (1) |
| 7 | 1 | 5 (1) | 2 (1) | 5 | 5 (2), 4 (3) | 2 (2), 3 (3) |
| 8 | 24 | 5 (24) | 3 (24) | 11 | 5 (11) | 3 (11) |

| | | |
|---|---|---|
| HR+^{‡} | 31 (74%) | 24 (57%) |

*The number of specimens is listed in the parenthesis after the Allred PS or IS.
^{†}Total number and percentage of hormone receptor negative (HR−) specimens.
^{‡}Total number and percentage of hormone receptor positive (HR+) specimens.

TABLE 11
Distributions of IHC Allred PS, IS, and TS for ER and PR
of sample set 3 (for both the 7500 and 7900 systems)

| Allred | ER (n = 291) | | | PR (n = 279) | | |
|---|---|---|---|---|---|---|
| TS | No. | Allred PS* | Allred IS* | No. | Allred PS* | Allred IS* |
| 0 | 6 | 0 (6) | 0 (6) | 17 | 0 (17) | 0 (17) |
| 2 | 2 | 1 (2) | 1 (2) | 9 | 1 (9) | 1 (9) |
| HR−^{†} | 8 (3%) | | | 26 (9%) | | |
| 3 | 1 | 2 (1) | 1 (1) | 35 | 2 (35) | 1 (35) |
| 4 | 2 | 3 (2) | 1 (2) | 26 | 3 (19), 2 (7) | 1 (19), 2 (7) |
| 5 | 6 | 4 (6) | 1 (6) | 49 | 4 (39), 3 (9), 2 (1) | 1 (39), 2 (9), 3 (1) |
| 6 | 28 | 5 (27), 4 (1) | 1 (27), 2 (1) | 44 | 5 (27), 4 (17) | 1 (27), 2 (17) |
| 7 | 109 | 5 (108), 4 (1) | 2 (108), 3 (1) | 53 | 5 (52), 4 (1) | 2 (52), 3 (1) |
| 8 | 137 | 5 (137) | 3 (137) | 46 | 5 (46) | 3 (46) |

| | | |
|---|---|---|
| HR+^{‡} | 283 (97%) | 253 (91%) |

*The number of specimens is listed in the parenthesis after the Allred PS or IS.
^{†}Total number and percentage of hormone receptor negative (HR−) specimens.
^{‡}Total number and percentage of hormone receptor positive (HR+) specimens.

TABLE 12
RNA samples used for determining normalization factor

From Ambion (Austin, TX):
cervix (adenocarcinoma)
epithelial carcinoma cell line A431
erythromyeloblastoid leukemia cell line K562
promyelocytic leukemia cell line HL-60
prostate cancer cell line PC3
T cell lymphoblast-like cell line Jurkat
Muscle
From BioChain (Hayward, CA):
adipose
breast
esophagus
fetal umbilical cord
heart (left atrium)
heart (left ventricle)
heart (right ventricle)
heart (pericardium)
liver
pancreas
stomach
From Stratagene (La Jolla, CA):
universal human reference RNA
breast
colon (adenocarcinoma)
colon (adult male)
colon (female)
erythromyeloblastoid leukemia cell line K562
erythromyeloblastoid leukemia cell line K562 (PMA treated)
ileum (chronic inflammation)
lung
ovary
prostate
thyroid
From Clontech (Mountain View, CA):
adrenal gland
bladder
bone marrow
fetal brain
fetal heart
fetal kidney
fetal liver
fetal spleen
fetal thymus
heart
heart (aorta)
heart (myocardial infarction)
heart (post myocardial infarction)
epithelial carcinoma cell line
HeLaS3
kidney
mammary gland
muscle
placenta
prostate
salivary gland
small intestine
spinal cord
spleen
thymus
thyroid
tonsil
trachea
whole brain

TABLE 13
TaqMan ® probes for mERPR RT-PCR assay
(such as for the 7900 system)

| Gene | | |
|---|---|---|
| Symbol | Reporter | Probe Sequence (5′→3′)* |
| ESR1 | 6FAM | TGTGCCTCAAATCTA (SEQ |
| | | ID NO:3) |
| PGR | VIC | TGACAGCCTGATGCTTCAT |
| | | (SEQ ID NO:6) |
| NUP214 | NED or TET | TCAGGAAATTCGGCGCCTT |
| | | (SEQ ID NO:12) |
| PPIG | NED or TET | ATGGTTCACAGTTCTTC |
| | | (SEQ ID NO:15) |

* TaqMan ® probes can have minor-groove binder and non-fluorescent quencher at 3′ termini.

TABLE 14
Classification of ER status of the discovery and validation sets using
the ΔΔC_{T} cutoff-point and clustering methods (7900 system)

| | Discovery (n = 67) | | | | | Validation (n = 270) | | | | |
|---|---|---|---|---|---|---|---|---|---|---|
| Allred | IHC | ΔΔC_{T} cutoff-point | | Clustering | | IHC | ΔΔC_{T} cutoff-point | | Clustering | |
| TS* | (% of total) | ER+ | ER− | ER+ | ER− | (% of total) | ER+ | ER− | ER+ | ER− |
| 0 | 17 | 2 | 15 | 1 | 16 | 4 | 1 | 3 | 0 | 4 |
| 2 | 0 | 0 | 0 | 0 | 0 | 2 | 0 | 2 | 0 | 2 |
| ER−^{†} | 17 (25%) | 2 | 15 | 1 | 16 | 6 (2%) | 1 | 5 | 0 | 6 |
| 3 | 2 | 0 | 2 | 0 | 2 | 1 | 0 | 1 | 0 | 1 |
| 4 | 0 | 0 | 0 | 0 | 0 | 2 | 2 | 0 | 1 | 1 |
| 5 | 3 | 3 | 0 | 3 | 0 | 5 | 5 | 0 | 3 | 2 |
| 6 | 2 | 2 | 0 | 2 | 0 | 27 | 27 | 0 | 26 | 1 |
| 7 | 12 | 12 | 0 | 12 | 0 | 102 | 102 | 0 | 101 | 1 |
| 8 | 31 | 31 | 0 | 31 | 0 | 127 | 127 | 0 | 127 | 0 |
| ER+^{‡} | 50 (75%) | 48 | 2 | 48 | 2 | 264 (98%) | 263 | 1 | 258 | 6 |

*Allred total score.
^{†}Total number of specimens with Allred TS0 and TS2 in each set.
^{‡}Total number of specimens with Allred TS3 to TS8 in each set.

TABLE 15
Summary of the performance of ER classification (7900 system)

| | Discovery (n = 67) | | Validation (n = 270) | |
|---|---|---|---|---|
| | ΔΔC_{T} cutoff-point | Clustering | ΔΔC_{T} cutoff-point | Clustering |
| | (95% CI) | (95% CI) | (95% CI) | (95% CI) |
| Sensitivity | 0.96 (0.86-1.00) | 0.96 (0.86-1.00) | 0.996 (0.98-1.00) | 0.98 (0.95-0.99) |
| Specificity | 0.88 (0.64-0.99) | 0.94 (0.71-1.00) | 0.83 (0.36-1.00) | 1.00 (0.54-1.00) |
| PPV | 0.96 (0.86-1.00) | 0.98 (0.89-1.00) | 0.996 (0.98-1.00) | 1.00 (0.99-1.00) |
| NPV | 0.88 (0.64-0.99) | 0.89 (0.65-0.99) | 0.83 (0.36-1.00) | 0.50 (0.21-0.79) |
| Accuracy | 0.94 (0.85-0.98) | 0.96 (0.87-0.99) | 0.99 (0.97-1.00) | 0.98 (0.95-0.99) |
| Kappa | 0.842 (0.693-0.992) | 0.884 (0.756-1.00) | 0.830 (0.597-1.000) | 0.657 (0.401-0.912) |

TABLE 16
Classification of PR status of the discovery and validation sets using
the ΔΔC_{T} cutoff-point and clustering methods (7900 system)

| | Discovery (n = 67) | | | | | Validation (n = 261) | | | | |
|---|---|---|---|---|---|---|---|---|---|---|
| Allred | IHC | ΔΔC_{T} cutoff-point | | Clustering | | IHC | ΔΔC_{T} cutoff-point | | Clustering | |
| TS* | (% of total) | PR+ | PR− | PR+ | PR− | (% of total) | PR+ | PR− | PR+ | PR− |
| 0 | 20 | 1 | 19 | 1 | 19 | 15 | 1 | 14 | 1 | 14 |
| 2 | 0 | 0 | 0 | 0 | 0 | 9 | 1 | 8 | 1 | 8 |
| PR−^{†} | 20 (30%) | 1 | 19 | 1 | 19 | 24 (9%) | 2 | 22 | 2 | 22 |
| 3 | 1 | 0 | 1 | 0 | 1 | 31 | 18 | 13 | 19 | 12 |
| 4 | 3 | 2 | 1 | 2 | 1 | 25 | 21 | 4 | 22 | 3 |
| 5 | 11 | 8 | 3 | 8 | 3 | 46 | 46 | 0 | 46 | 0 |
| 6 | 11 | 10 | 1 | 10 | 1 | 44 | 44 | 0 | 44 | 0 |
| 7 | 7 | 7 | 0 | 7 | 0 | 47 | 46 | 0 | 46 | 0 |
| 8 | 14 | 14 | 0 | 14 | 0 | 44 | 45 | 0 | 45 | 0 |
| PR+^{‡} | 47 (70%) | 41 | 6 | 41 | 6 | 237 (91%) | 220 | 17 | 222 | 15 |

*Allred total score.
^{†}Total number of specimens with Allred TS0 and TS2 in each set.
^{‡}Total number of specimens with Allred TS3 to TS8 in each set.

TABLE 17
Summary of the performance of PR classification (7900 system)

| | Discovery (n = 67) | | Validation (n = 261) | |
|---|---|---|---|---|
| | ΔΔC_{T} cutoff-point | Clustering | ΔΔC_{T} cutoff-point | Clustering |
| | (95% CI) | (95% CI) | (95% CI) | (95% CI) |
| Sensitivity | 0.87 (0.74-0.95) | 0.87 (0.74-0.95) | 0.93 (0.89-0.96) | 0.94 (0.90-0.96) |
| Specificity | 0.95 (0.75-1.00) | 0.95 (0.75-1.00) | 0.92 (0.73-0.99) | 0.92 (0.73-0.99) |
| PPV | 0.98 (0.87-1.00) | 0.98 (0.87-1.00) | 0.99 (0.97-1.00) | 0.99 (0.97-1.00) |
| NPV | 0.76 (0.55-0.91) | 0.76 (0.55-0.91) | 0.56 (0.40-0.72) | 0.59 (0.42-0.75) |
| Accuracy | 0.90 (0.80-0.96) | 0.90 (0.80-0.96) | 0.93 (0.89-0.96) | 0.93 (0.90-0.96) |
| Kappa | 0.767 (0.607-0.928) | 0.767 (0.607-0.928) | 0.660 (0.520-0.800) | 0.686 (0.548-0.824) |

TABLE 18
7900 and 7500 system comparison

| | Square of Pearson's | | |
|---|---|---|---|
| | correlation coefficient | Concordance of Status | |
| | (r²) of ΔΔC_{T} values | ΔΔC_{T} method* | clustering method* |
| ER | 0.9783 (p < 0.0001) | 100% (337/337) | 99.4% (335/337) |
| PR | 0.9698 (p < 0.0001) | 96.3% (316/328) | 98.8% (324/328) |

*The ΔΔC_{T} cutoff points and the clustering analysis parameters for ER and PR classifications were derived from the discovery results obtained from each instrument platform. For the 7500 system, the ΔΔC_{T} cutoff points were 1.5 and 0.5 for ER and PR classifications, respectively. For the 7900 system, a ΔΔC_{T} cutoff point of 1.0 was used for both ER and PR classifications. Similarly, the clustering analysis parameters were determined independently for the two instrument platforms. The concordance of hormonal receptor status between the two platforms is reported for both discovery and validation sets.

TABLE 19
Genes comprising the 14-gene metastasis prognostic panel and endogenous controls.

| Gene | MS constant ai | RefSeq | Description | Reference Citation |
|---|---|---|---|---|
| CENPA | 0.29 | NM_001809 | centromere protein A, | Black, B. E., Foltz, D. R., et al., Nature |
| | | | 17 kDa | 430(6999): 578-582 (2004) |
| PKMYT1 | 0.29 | NM_004203 | membrane-associated | Bryan, B. A., Dyson, O. F. et al., J. Gen. |
| | | | tyrosine- and | Virol. 87 (PT 3), 519-529 (2006) |
| | | | thereonine-specific | |
| | | | cdc2-inhibitory kinase | |
| MELK | 0.29 | NM_014791 | maternal embryonic | Beullens, M., Vancauwenbergh, S. et al., |
| | | | leucine zipper kinase | J. Biol. Chem. 280 (48), 40003-40011 |
| | | | | (2005) |
| MYBL2 | 0.29 | NM_002466 | v-myb myeloblastosis | Bryan, B. A., Dyson, O. F. et al., J. Gen. |
| | | | viral oncogene | Virol. 87 (PT 3), 519-529 (2006) |
| | | | homolog (avian)-like 2 | |
| BUB1 | 0.27 | NM_004336 | BUB1 budding | Morrow, C. J., Tighe, A. et al., J. Cell. Sci. |
| | | | uninhibited by | 118 (PT 16), 3639-3652 (2005) |
| | | | benzimidazoles 1 | |
| | | | homolog | |
| RACGAP1 | 0.29 | NM_013277 | Rac GTPase activating | Niiya, F., Xie, X. et al., J. Biol. Chem. 280 |
| | | | protein 1 | (43), 36502-36509 (2005) |
| TK1 | 0.27 | NM_003258 | thymidine kinase 1, | Karbownik, M., Brzezianska, E. et al., |
| | | | soluble | Cancer Lett. 225 (2), 267-273 (2005) |
| UBE2S | 0.27 | NM_014501 | ubiquitin-conjugating | Liu, Z., Diaz, L. A. et al., J. Biol. Chem. |
| | | | enzyme E2S | 267 (22), 15829-15835 (1992) |
| C16orf61 | 0.22 | NM_020188 | DC13 protein | Gu, Y., Peng, Y. et al., Direct Submission, |
| (DC13) | | AF201935 | | Submitted (05 NOV. 1999) Chinese |
| | | | | National Human Genome Center at |
| | | | | Shanghai, 351 Guo Shoujing Road, |
| | | | | Zhangjiang Hi-Tech Park, Pudong, |
| | | | | Shanghai 201203, P. R. China |
| RFC4 | 0.25 | NM_002916 | replication factor C | Gupte, R. S., Weng, Y. et al., Cell Cycle 4 |
| | | | (activator 1) 4, 37 kDa | (2), 323-329 (2005) |
| PRR11 | 0.26 | NM_018304 | proline rich 11 | Weinmann, A. S., Yan, P. S. et al., Genes |
| (FLJ11029) | | | | Dev. 16 (2), 235-244 (2002) |
| DIAPH3 | 0.23 | NM_030932 | diaphanous homolog 3 | Katoh, M. and Katoh, M., Int. J. Mol. |
| | | | (Drosophila) | Med. 13 (3), 473-478 (2004) |
| ORC6L | 0.28 | NM_014321 | origin recognition | Sibani, S., Price, G. B. et al., Biochemistry |
| | | | complex, subunit 6 | 44 (21), 7885-7896 (2005) |
| | | | homolog-like (yeast) | |
| CCNB1 | 0.23 | NM_031966 | cyclin B1 | Zhao, M., Kim, Y. T. et al., |
| | | | | Exp Oncol 28 (1), 44-48 (2006) |
| PPIG | EC | NM_004792 | peptidylprolyl | Lin, C. L., Leu, S. et al., Biochem. Biophys. |
| | | | isomerase G | Res. Commun. 321 (3), 638-647 (2004) |
| NUP214 | EC | NM_005085 | nucleoporin 214 kDa | Graux, C., Cools, J. et al., Nat. Genet. 36 |
| | | | | (10), 1084-1089 (2004) |
| SLU7 | EC | NM_006425 | step II splicing factor | Shomron, N., Alberstein, M. et al., J. Cell. |
| | | | | Sci. 118 (PT 6), 1151-1159 (2005) |

“Ref Seq” = NCBI reference sequence for one variant of the specified gene “EC” = Endogenous Control

TABLE 20
Exemplary fluorescent dyes.

| Dye Name | Absorption (nm) | Emission(nm) |
|---|---|---|
| SYBR | 497 | 520 |
| FAM | 495 | 520 |
| TET | 521 | 536 |
| CAL Fluor Gold 540 | 522 | 544 |
| JOE | 520 | 548 |
| VIC | 538 | 554 |
| HEX | 535 | 556 |
| MAX557 | | 557 |
| CAL Fluor Orange 560 | 538 | 560 |
| QUASAR 570 | 548 | 566 |
| Cy3 | 550 | 570 |
| NED | | 573 |
| TAMRA | 555 | 576 |
| TRE | 555 | 580 |
| CAL Fluor Red 590 | 569 | 591 |
| PET | | 595 |
| Cy3.5 | 581 | 596 |
| ROX | 575 | 602 |
| Texas Red | 583 | 603 |
| CAL Fluor Red | 590 | 610 |
| TEX615 | | 615 |
| PHO | 595 | 617 |
| CAL Fluor Red 635 | 618 | 637 |
| NPR | 640 | 655 |
| TYE665 | | 665 |
| QUASAR 670 | 647 | 667 |
| Cy5 | 649 | 670 |
| Cy5.5 | 675 | 694 |

The fluorescent reporters used in certain exemplary mERPR+HER2 assays are highlighted above (in bold). Other dyes, or combinations of dyes, with distinguishable fluorescent emissions, including but not limited to any of the dyes listed above in Table 20, may be used in any of the assays disclosed herein. For example, if expression detection of one or more other genes of interest (and/or other control genes such as other housekeeping genes) are added to an assay, then dyes such as any of the above can be used for detection of these other genes.

TABLE 21
Example of parameters used for clustering analysis.

| | | |
|---|---|---|
| ER | PR | HER2 |

Prior probabilities

| | | | | | |
|---|---|---|---|---|---|
| p_{ER-} | 0.2909894 | p_{PR-} | 0.3633805 | p_{HER2 norm} | 0.8470969 |
| p_{ER+} | 0.7090106 | p_{PR+} | 0.6366195 | p_{HER2 over} | 0.1529031 |

Means

| | | | | | |
|---|---|---|---|---|---|
| u_{ER-} | 0.09678518 | u_{PR-} | −0.7653664 | u_{HER2 norm} | 2.348768 |
| u_{ER+} | 4.92452139 | u_{PR+} | 3.9643629 | u_{HER2 over} | 6.200021 |

Variance

| | | | | | |
|---|---|---|---|---|---|
| v_{ER-} | 1.285882 | v_{PR-} | 3.679639 | v_{HER2 norm} | 1.139182 |
| v_{ER+} | 1.285882 | v_{PR+} | 3.679639 | v_{HER2 over} | 1.139182 |

What is claimed is:

1. A method of determining estrogen receptor (ER) and progesterone receptor (PR) status in a sample from a human, comprising simultaneously detecting ESR1 mRNA and PGR mRNA in a multiplex assay, and determining ER and PR status based on expression levels of said ESR1 mRNA and PGR mRNA.

2. The method of claim 1, further comprising detecting ERBB2 mRNA in said multiplex assay, and determining ERBB2 status based on expression levels of said ERBB2 mRNA.

3. The method of claim 1, further comprising detecting mRNA of at least one control gene to which ESR1 mRNA and PGR mRNA levels are normalized against.

4. The method of claim 3, wherein the control gene comprises at least one of NUP214 and PPIG.

5. The method of claim 1, wherein the multiplex assay is a TaqMan® assay.

6. The method of claim 1, wherein the human has breast cancer.

7. The method of claim 1, wherein the sample is a formalin-fixed paraffin-embedded (FFPE) sample or a frozen sample.

8. The method of claim 7, wherein the FFPE sample is a breast tumor tissue sample.

9. The method of claim 1, wherein the mRNA is reverse transcribed to cDNA and detected by PCR amplification of said cDNA.

10. The method of claim 9, wherein the mRNA is enriched prior to reverse transcription and PCR amplification.

11. The method of claim 2, wherein the mRNA of ESR1, PGR, and ERBB2 is reverse transcribed and amplified by at least one primer for each gene as presented in Table 2, SEQ ID NOS: 1-2, 4-5, and 7-8.

12. The method of claim 11, wherein the mRNA of ESR1, PGR, and ERBB2 is detected by a probe for each gene as presented in Table 2, SEQ ID NOS:3, 6, and 9.

13. The method of claim 4, wherein the mRNA of NUP214 and PPIG is reverse transcribed and amplified by the primers for each gene as presented in Table 2, SEQ ID NOS:10-11 and 13-14.

14. The method of claim 13, wherein the mRNA of NUP214 and PPIG is detected by a probe for each gene as presented in Table 2, SEQ ID NOS:12 and 15.

15. The method of claim 1, wherein the expression level of each mRNA is calculated by the Δ(ΔC_{T}) method, wherein:
Δ(ΔCt)=(−1)×(Ct_{GOI}−Ct_{EC})_{test RNA}−(Ct_{GOI}−Ct_{EC})_{ref RNA}
where Ct is the PCR threshold cycle of exponential target amplification, GOI=gene of interest, EC=endogenous control, test RNA=patient sample RNA, ref RNA=reference RNA.

16. The method of claim 1, further comprising determining whether the human will benefit from a treatment based on at least one of the ER and PR status of the human.

17. The method of claim 16, wherein the human has breast cancer, and wherein the treatment is a hormonal therapy.

18. The method of claim 17, wherein the hormonal therapy is a selective estrogen receptor modulator (SERM).

19. The method of claim 18, wherein the selective estrogen receptor modulator is tamoxifen.

20. The method of claim 2, further comprising determining whether the human will benefit from a treatment based on at least one of the ER, PR, and ERBB2 status of the human.

21. The method of claim 20, wherein the human has breast cancer, and wherein the treatment is a therapeutic agent that targets the Her-2 receptor.

22. The method of claim 21, wherein the therapeutic agent is Trastuzumab (Herceptin®).

23. The method of claim 1, further comprising determining risk of tumor metastasis in a breast cancer patient, the method comprising detecting mRNA of genes CENPA, PKMYT1, MELK, MYBL2, BUB1, RACGAP1, TK1, UBE2S, C16orf61 (DC13), RFC4, PRR11, DIAPH3, ORC6L, and CCNB1, and predicting risk of tumor metastasis based on expression levels of said mRNA.

24. The method of claim 23, further comprising detecting ERBB2 mRNA.

25. The method of claim 23, further comprising detecting mRNA of at least one control gene.

26. The method of claim 25 wherein the control gene comprises at least one of NUP214, PPIG, and SLU7.

27. A kit comprising reagents for detecting ESR1 mRNA and PGR mRNA, enzyme, and a buffer.

28. The kit of claim 27, further comprising reagents for detecting ERBB2 mRNA.

29. The kit of claim 27, further comprising reagents for detecting mRNA of at least one control gene.

30. The kit of claim 29, wherein the control gene comprises at least one of NUP214 and PPIG.

31. The kit of claim 27, wherein the reagents are for a TaqMan® assay.

32. The kit of claim 28, wherein the reagents comprise at least one primer for amplifying at least one of ESR1, PGR, and ERBB2, wherein the primer is presented in Table 2, SEQ ID NOS:1-2, 4-5, and 7-8.

33. The kit of claim 28, wherein the reagents comprise at least one probe for detecting at least one of ESR1, PGR, and ERBB2, wherein the probe is presented in Table 2, SEQ ID NOS:3, 6, and 9.

34. The kit of claim 30, wherein the reagents comprise at least one primer for amplifying at least one of NUP214 and PPIG, wherein the primer is presented in Table 2, SEQ ID NOS: 10-11 and 13-14.

35. The kit of claim 30, wherein the reagents comprise at least one probe for detecting at least one of NUP214 and PPIG, wherein the probe is presented in Table 2, SEQ ID NOS:12 and 15.

36. The kit of claim 27, further comprising reagents for detecting mRNA of genes CENPA, PKMYT1, MELK, MYBL2, BUB1, RACGAP1, TK1, UBE2S, C16orf61 (DC13), RFC4, PRR11, DIAPH3, ORC6L, and CCNB1.

37. The kit of claim 36, further comprising reagents for detecting ERBB2 mRNA.

38. The kit of claim 36, further comprising reagents for detecting mRNA of at least one control gene.

39. The kit of claim 38, wherein the control gene comprises at least one of NUP214, PPIG, and SLU7.

40. The method of claim 3, which comprises detecting mRNA of a plurality of control genes, and wherein probes for detecting each of the control genes are labeled with the same dye.

41. The method of claim 40, wherein the control genes comprise NUP214 and PPIG, and wherein probes for detecting NUP214 and PPIG are each labeled with the same dye.

42. The kit of claim 29, wherein the reagents are for detecting mRNA of a plurality of control genes, and wherein probes for detecting each of the control genes are labeled with the same dye.

43. The kit of claim 42, wherein the control genes comprise NUP214 and PPIG, and wherein probes for detecting NUP214 and PPIG are each labeled with the same dye.


SEQUENCE LISTING (29 sequences)

SEQ ID NO: 1 - DNA, 19 bp, Homo sapiens
tctgcagggagaggagttt

SEQ ID NO: 2 - DNA, 22 bp, Homo sapiens
ggtccttctcttccagagactt

SEQ ID NO: 3 - DNA, 15 bp, Homo sapiens
tgtgcctcaaatcta

SEQ ID NO: 4 - DNA, 22 bp, Homo sapiens
tcgagtcattacctcagaagat

SEQ ID NO: 5 - DNA, 21 bp, Homo sapiens
cccacaggtaaggacaccata

SEQ ID NO: 6 - DNA, 19 bp, Homo sapiens
tgacagcctgatgcttcat

SEQ ID NO: 7 - DNA, 19 bp, Homo sapiens
cagccctggtcacctacaa

SEQ ID NO: 8 - DNA, 18 bp, Homo sapiens
gggacaggcagtcacaca

SEQ ID NO: 9 - DNA, 18 bp, Homo sapiens
tgagtccatgcccaatcc

SEQ ID NO: 10 - DNA, 24 bp, Homo sapiens
catttgctttataaaagaccactg

SEQ ID NO: 11 - DNA, 22 bp, Homo sapiens
ccactccaagtctagaacatca

SEQ ID NO: 12 - DNA, 19 bp, Homo sapiens
tcaggaaattcggcgcctt

SEQ ID NO: 13 - DNA, 19 bp, Homo sapiens
gccaacagagggaaggata

SEQ ID NO: 14 - DNA, 21 bp, Homo sapiens
gaggagttggtttcgttgtta

SEQ ID NO: 15 - DNA, 17 bp, Homo sapiens
atggttcacagttcttc

SEQ ID NO: 16 - DNA, 6330 bp, Homo sapiens
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 17 - DNA, 1366 bp, Homo sapiens
aggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggacataacgactatatgtgtccagccaccaaccagtgcaccattgataaaaacaggaggaagagctgccaggcctgccggctccgcaaatgctacgaagtgggaatgatgaaaggtggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggc

SEQ ID NO: 18 - DNA, 1249 bp, Homo sapiens
aggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggc

SEQ ID NO: 19 - DNA, 1128 bp, Homo sapiens
aggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccaccttctagaatgtgcctggctagagatcctgatgattggtctcgtctggcgctccatggagcacccagtgaagctactgtttgctcctaacttgctcttggacaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggc

SEQ ID NO: 20 - DNA, 13037 bp, Homo sapiens
agtccacagctgtcactaatcggggtaagccttgttgtatttgtgcgtgtgggtggcattctcaatgagaactagcttcacttgtcatttgagtgaaatctacaacccgaggcggctagtgctcccgcactactgggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagctgaccagcgccgccctcccccgcccccgacccaggaggtggagatccctccggtccagccacattcaacacccactttctcctccctctgcccctatattcccgaaaccccctcctccttcccttttccctcctcctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggtatgtctttttgttttggtcaggattatgaggtcttgagtttttataatgttcttctgaaagccttacatttataacatcatagtgtgtaaatttaaaagaaaaattgtgaggttctaattattttcttttataaagtataattagaatgtttaactgttttgtttacccatattttcttgaagaatttacaagattgaaaaagtactaaaattgttaaagtaaactatcttatccatattatttcataccatgtaggtgaggatttttaacttttgcatctaacaaatcatcgacttaagagaaaaaatcttacatgtaataacacaaagctattatatgttatttctaggtaactccctttgtgtcaattatatttccaaaaatgaacctttaaaatggtatgcaaaattttgtctatatatatttgtgtgaggaggaaattcataactttcctcagattttcaaaagtatttttaatgcaaaaaatgtagaaagagtttaaaaccactaaaatagattgatgttcttcaaactaggcaaaacaactcatatgttaagaccattttccagattggaaacacaaatctcttaggaagttaataagtagattcatatcattatgcaaatagtattgtgggttttgtaggtttttaaaataaccttttttggggagagaattgtcctctaatgaggtattgcgagtggacataagaaatcagaagattatggcctaactgtactccttaccaactgtggcatgctgaaagttagtcactcttactgattctcaattctctcacctttgaaagtagtaaaatatctttcctgccaattgctcctttgggtcagagcttattaacatcttttcaaatcaaaggaaagaagaaagggagaggaggaggagggaggtatcaattcacatacctttctcctctttatcctccactatcatgaattcatattatgtttcagccatgcaaatctttttaccatgaaatttcttccagaattttccccctttgacacaaattccatgcatgtttcaaccttcgagactcagccaaatgtcatttctgtaaaatcttccctgagtcttccaagcagtaatttgccttctcctagagtttacctgccattttgtgcacatttgagttacagtagcatgttattttacaattgtgactctcctgggagtctgggagccatataaagtggtcaatagtgtttgctgactgagagttgaatgacattttctctctgtcttggtattactgtagatttcgatcattctttggttacatttctgcatatttctgtacccatgactttatcactttcttctcccatgctttatctccatcaattatcttcattacttttaaattttccacctttgcttcctactttgtgagatctctccctttactgactataacatagaagaatagaagtgtattttatgtgtcttaaggacaatactttagattccttgttctaagtttttaaactgaatgaatggaatattatttctctccctaagcaaaattccacaaaacaattatttcttatgtttatgtagccttaaattgttttgtactgtaaacctcagcataaaaactttcttcatttctaatttcattcaacaaatattgattgaatacctggtattagcacaagaaaaatgtgctaataagccttatgagaatttggagctgaagaaagacatataactcaggaaagttacagtccagtagtaggtataaattacagtgcctgataaataggcattttaatatttgtacactcaacgtatactaggtaggtgcaaaacatttacatataattttactgatacccatgcagcacaaaggtactaactttaaatattaaataacacctttatgtgtcagtaattcatttgcattaaatcttattgaaaaggctttcaatatattttccccacaaatgtcatcccaagaaaaaagtatttttaacatctcccaaatataatagttacaggaaatctacctctgtgagagtgacacctctcagaatgaactgtgtgacacaagaaaatgaatgtaggtctatccaaaaaaaaccccaagaaacaaaaacaatattattagccctttatgcttaagtgatggactcagggaacagttgatgttgtgatcattttattatctgattcttgttactttgaattaaaccaatattttgatgatataaatcatttccaccagcatatatttaatttccataataactttaaaattttctaatttcactcaactatgagggaatagaatgtggtggccacaggtttggcttttgttaaaatgtttgatatcttcgatgttgatctctgtctgcaatgtagatgtctaaacactaggatttaatatttaaggctaagctttaaaaataaagtacctttttaaaaagaatatggcttcaccaaatggaaaatacctaatttctaaatctttttctctacaaagtcctatctactaatgtctccattactatttagtcatcataaccattatcttcattttacatgtcgtgttctttctggtagctctaaaatgacactaaatcataagaagacaggttacatatcaggaaatacttgaaggttactgaaatagattcttgagttaatgaaaatattttctgtaaaaaggtttgaaaagccatttgagtctaaagcattatacctccattatcagtagttatgtgacaattgtgtgtgtgtttaatgtttaaagatgtggcactttttaataaggcaatgctatgctattttttcccatttaacattaagataatttattgctatacagatgatatggaaatatgatgaacaatattttttttgccaaaactatgccttgtaagtagccatggaatgtcaacctgtaacttaaattatccacagatagtcatgtgtttgatgatgggcactgtggagataactgacataggactgtgccccccttctctgccacttactagctggatgagattaagcaagtcatttaactgctctgattaaacctgcctttcccaagtgctttgtaatgaatagaaatggaaaccaaaaaaaacgtatacaggccttcagaaatagtaattgctactattttgttttcattaagccatagttctggctataattttatcaaactcaccagctatattctacagtgaaagcaggattctagaaagtctcactgttttatttatgtcaccatgtgctatgatatatttggttgaattcatttgaaattagggctggaagtattcaagtaatttcttctgctgaaaaaatacagtgttttgagtttagggcctgttttatcaaagttctaaagagcctatcactcttccattgtagacattttaaaataatgacactgattttaacatttttaagtgtctttttagaacagagagcctgactagaacacagcccctccaaaaacccatgctcaaattatttttactatggcagcaattccacaaaagggaacaatgggtttagaaattacaatgaagtcatcaacccaaaaaacatccctatccctaagaaggttatgatataaaatgcccacaagaaatctatgtctgctttaatctgtcttttattgctttggaaggatggctattacatttttagtttttgctgtgaatacctgagcagtttctctcatccatacttatccttcacacatcagaagtcaggatagaatatgaatcattttaaaaacttttacaactccagagccatgtgcataagaagcattcaaaacttgccaaaacatacattttttttcaaatttaaagatactctatttttgtattcaatagctcaacaactgtggtccccactgataaagtgaagtggacaaggagacaagtaatggcataagtttgtttttcccaaagtatgcctgttcaatagccattggatgtgggaaatttctacatctcttaaaattttacagaaaatacatagccagatagtctagcaaaagttcaccaagtcctaaattgcttatccttacttcactaagtcatgaaatcattttaatgaaaagaacatcacctaggttttgtggtttctttttttcttattcatggctgagtgaaaacaacaatctctgtttctccctagcatctgtggactatttaatgtaccattattccacactctatggtccttactaaatacaaaattgaacaaaaagcagtaaaacaactgactcttcacccatattataaaatataatccaagccagattagtcaacatccataagatgaatccaagctgaactgggcctagattattgagttcaggttggatcacatccctatttattaataaacttaggaaagaaggccttacagaccatcagttagctggagctaatagaacctacacttctaaagttcggcctagaatcaatgtggccttaaaagctgaaaagaagcaggaaagaacagttttcttcaataatttgtccaccctgtcactggagaaaatttaagaatttgggggtgttggtagtaagttaaacacagcagctgttcatggcagaaattattcaatacataccttctctgaatatcctataaccaaagcaaagaaaaacaccaaggggtttgttctcctccttggagttgacctcattccaaggcagagctcaggtcacaggcacaggggctgcgcccaagcttgtccgcagccttatgcagctgtggagtctggaagactgttgcaggactgctggcctagtcccagaatgtcagcctcattttcgatttactggctcttgttgctgtatgtcatgctgaccttattgttaaacacaggtttgtttgctttttttccactcatggagacatgggagaggcattatttttaagctggttgaaagctttaaccgataaagcatttttagagaaatgtgaatcaggcagctaagaaagcatactctgtccattacggtaaagaaaatgcacagattattaactctgcagtgtggcattagtgtcctggtcaatattcggatagatatgaataaaatatttaaatggtattgtaaatagttttcaggacatatgctatagcttatttttattatcttttgaaattgctcttaatacatcaaatcctgatgtattcaatttatcagatataaattattctaaatgaagcccagttaaatgtttttgtcttgtcagttatatgttaagtttctgatctctttgtctatgacgtttactaatctgcatttttactgttatgaattattttagacagcagtggtttcaagctttttgccactaaaaataccttttattttctcctcccccagaaaagtctataccttgaagtatctatccaccaaactgtacttctattaagaaatagttattgtgttttcttaatgttttgttattcaaagacatatcaatgaaagctgctgagcagcatgaataacaattatatccacacagatttgatatattttgtgcagccttaacttgatagtataaaatgtcattgctttttaaataatagttagtcaatggacttctatcatagctttcctaaactaggttaagatccagagctttggggtcataatatattacatacaattaagttatctttttctaagggctttaaaattcatgagaataaccaaaaaaggtatgtggagagttaatacaaacataccatattcttgttgaaacagagatgtggctctgcttgttctccataaggtagaaatactttccagaatttgcctaaactagtaagccctgaatttgctatgattagggataggaagagattttcacatggcagactttagaattcttcactttagccagtaaagtatctccttttgatcttagtattctgtgtattttaacttttctgagttgtgcatgtttataagaaaaatcagcacaaagggtttaagttaaagcctttttactgaaatttgaaagaaacagaagaaaatatcaaagttctttgtattttgagaggattaaatatgatttacaaaagttacatggagggctctctaaaacattaaattaattattttttgttgaaaagtcttactttaggcatcattttattcctcagcaactagctgtgaagcctttactgtgctgtatgccagtcactctgctagattgtggagattaccagtgttcccgtcttctccgagcttagagttggatggggaataaagacaggtaaacagatagctacaatattgtactgtgaatgcttatgctggaggaagtacagggaactattggagcacctaagaggagcacctaccttgaatttaggggttagcagaggcatcctgaaaaaagtcaaagctaagccacaatctataagcagtttaggaattagcagaacgtgcgtggtgaggagatgccaaaggcaagaagagaagagtattccaaacaggagggattccaaagagagaagagtatcccaaacaacatttgcacaaacctgatggggagagagaatgtggggtggggatggatgatgagactgaagaagaaagccaggtctagataatcagtggccttgtacaccatgttaaagagtgtagacttgattctgttgtaaacaggaaagcagcacaattcatatgaatattttagaagactcccactggaatatggagaataaagttggagatgactaatcctggaagcagggagaacatttttgaggaagttgcactattttggtgaaaatgatgatcataaacatgaagaattgtaggtgatcatgacctcctctctaattttccagaagggttttggaagatataacataggaacattgacaggactgacgaaaggagatgaaatacaccatataaattgtcaaacacaaggccagatgtctaattattttgcttatgtgttgaaattacaaatttttcatcaggaaaccaaaaactacaaaacttagttttcccaagtcccagaattctatctgtccaaacaatctgtaccactccacctatatccctacctttgcatgtctgtccaacctcaaagtccaggtctatacacacgggtaagactagagcagttcaagtttcagaaaatgagaaagaggaactgagttgtgctgaacccatacaaaataaacacattctttgtatagattcttggaacctcgagaggaattcacctaactcataggtatttgatggtatgaatccatggctgggctcggcttttaaaaagccttatctgggattccttctatggaaccaagttccatcaaagcccatttaaaagcctacattaaaaacaaaattcttgctgcattgtatacaaataatgatgtcatgatcaaataatcagatgccattatcaagtggaattacaaaatggtatacccactccaaaaaaaaaaaaaaagctaaattctcagtagaacattgtgacttcatgagccctccacagccttggagctgaggagggagcactggtgagcagtaggttgaagagaaaacttggcgcttaataatctatccatgttttttcatctaaaagagccttctttttggattaccttattcaatttccatcaaggaaattgttagttccactaaccagacagcagctgggaaggcagaagcttactgtatgtacatggtagctgtgggaaggaggtttctttctccaggtcctcactggccatacaccagtcccttgttagttatgcctggtcatagacccccgttgctatcatctcatatttaagtctttggcttgtgaatttatctattctttcagcttcagcactgcagagtgctgggactttgctaacttccatttcttgctggcttagcacattcctcataggcccagctcttttctcatctggccctgctgtggagtcaccttgccccttcaggagagccatggcttaccactgcctgctaagcctccactcagctgccaccacactaaatccaagcttctctaagatgttgcagactttacaggcaagcataaaaggcttgatcttcctggacttccctttacttgtctgaatctcacctccttcaactttcagtctcagaatgtaggcatttgtcctctttgccctacatcttccttcttctgaatcatgaaagcctctcacttcctcttgctatgtgctggaggcttctgtcaggttttagaatgagttctcatctagtcctagtagcttttgatgcttaagtccaccttttaaggatacctttgagatttagaccatgtttttcgcttgagaaagccctaatctccagacttgcctttctgtggatttcaaagaccaactgaggaagtcaaaagctgaatgttgactttctttgaacatttccgctataacaattccaattctcctcagagcaatatgcctgcctccaactgaccaggagaaaggtccagtgccaaagagaaaaacacaaagattaattatttcagttgagcacatactttcaaagtggtttgggtattcatatgaggttttctgtcaagagggtgagactcttcatctatccatgtgtgcctgacagttctcctggcactggctggtaacagatgcaaaactgtaaaaattaagtgatcatgtattttaacgatatcatcacatacttattttctatgtaatgttttaaatttcccctaacatactttgactgttttgcacatggtagatattcacatttttttgtgttgaagttgatgcaatcttcaaagttatctaccccgttgcttattagtaaaactagtgttaatacttggcaagagatgcagggaatctttctcatgactcacgccctatttagttattaatgctactaccctattttgagtaagtagtaggtccctaagtacattgtccagagttatacttttaaagatatttagccccatatacttcttgaatctaaagtcatacaccttgctcctcatttctgagtgggaaagacatttgagagtatgttgacaattgttctgaaggtttttgccaagaaggtgaaactgtcctttcatctgtgtatgcctggggctgggtccctggcagtgatggggtgacaatgcaaagctgtaaaaactaggtgctagtgggcacctaatatcatcatcatatacttattttcaagctaatatgcaaaatcccatctctgtttttaaactaagtgtagatttcagagaaaatattttgtggttcacataagaaaacagtctactcagcttgacaagtgttttatgttaaattggctggtggtttgaaatgaatcatcttcacataatgttttctttaaaaatattgtgaatttaactctaattcttgttattctgtgtgataataaagaataaactaatttcta

SEQ ID NO: 21 - DNA, 2365 bp, Homo sapiens
atgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggttttcgaaacttacatattgatgaccagataactctcattcagtattcttggatgagcttaatggtgtttggtctaggatggagatcctacaaacacgtcagtgggcagatgctgtattttgcacctgatctaatactaaatgattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtga

SEQ ID NO: 22 - DNA, 2392 bp, Homo sapiens
atgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggttttcgaaacttacatattgatgaccagataactctcattcagtattcttggatgagcttaatggtgtttggtctaggatggagatcctacaaacacgtcagtgggcagatgctgtattttgcacctgatctaatactaaatgagcagagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtga

SEQ ID NO: 23 - DNA, 2750 bp, Homo sapiens
atgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtga

SEQ ID NO: 24 - DNA, 4624 bp, Homo sapiens
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 25 - DNA, 4816 bp, Homo sapiens
gttcccggatttttgtgggcgcctgccccgcccctcgtccccctgctgtgtccatatatcgaggcgatagggttaagggaaggcggacgcctgatgggttaatgagcaaactgaagtgttttccatgatcttttttgagtcgcaattgaagtaccacctcccgagggtgattgcttccccatgcggggtagaacctttgctgtcctgttcaccactctacctccagcacagaatttggcttatgcctactcaatgtgaagatgatgaggatgaaaacctttgtgatgatccacttccacttaatgaatggtggcaaagcaaagctatattcaagaccacatgcaaagctactccctgagcaaagagtcacagataaaacgggggcaccagtagaatggccaggacaaacgcagtgcagcacagagactcagaccctggcagccatgcctgcgcaggcagtgatgagagtgacatgtactgttgtggacatgcacaaaagtgagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 26 - DNA, 6614 bp, Homo sapiens
ctgcgcgccgctggcgctgaggggaggaagtttgctgtcgagcggcctgggttccgtgggcaaggccgtgggaggcagcgttggctgcttcgacacactgagggcggcgcgatgggagacgagatggatgccatgattcccgagcgggagatgaaggattttcagtttagagcgctaaagaaggtgagaatctttgactcccctgaggaattgcccaaggaacgctcgagtctgcttgctgtgtccaacaaatatggtctggtcttcgctggtggagccagtggcttgcagatttttcctactaaaaatcttcttattcaaaataaacccggagatgatcccaacaaaatagttgataaagtccaaggcttgctagttcctatgaaattcccaatccatcacctggccttgagctgtgataacctcacactctctgcgtgcatgatgtccagtgaatatggttccattattgctttttttgatgttcgcacattctcaaatgaggctaaacagcaaaaacgcccatttgcctatcataagcttttgaaagatgcaggaggcatggtgattgatatgaagtggaaccccactgtcccctccatggtggcagtttgtctggctgatggtagtattgctgtcctgcaagtcacggaaacagtgaaagtatgtgcaactcttccttccacggtagcagtaacctctgtgtgctggagccccaaaggaaagcagctggcagtgggaaaacagaatggaactgtggtccagtatcttcctactttgcaggaaaaaaaagtcattccttgtcctccgttttatgagtcagatcatcctgtcagagttctggatgtgctgtggattggtacctacgtcttcgccatagtgtatgctgctgcagatgggaccctggaaacgtctccagatgtggtgatggctctactaccgaaaaaagaagaaaagcacccagagatatttgtgaactttatggagccctgttatggcagctgcacggagagacagcatcattactacctcagttacattgaggaatgggatttagtgctggcagcatctgcggcttcaacagaagttagtatccttgctcgacaaagtgatcagattaattgggaatcttggctactggaggattctagtcgagctgaattgcctgtgacagacaagagtgatgactccttgcccatgggagttgtcgtagactatacaaaccaagtggaaatcaccatcagtgatgaaaagactcttcctcctgctccagttctcatgttactttcaacagatggtgtgctttgtccattttatatgattaatcaaaatcctggggttaagtctctcatcaaaacaccagagcgactttcattagaaggagagcgacagcccaagtcaccaggaagtactcccactaccccaacctcctctcaagccccacagaaactggatgcttctgcagctgcagcccctgcctctctgccaccttcatcacctgctgctcccattgccactttttctttgcttcctgctggtggagcccccactgtgttctcctttggttcttcatctttgaagtcatctgctacggtcactggggagcccccttcatattccagtggctccgacagctccaaagcagccccaggccctggcccatcaaccttctcttttgttcccccttctaaagcctccctagcccccacccctgcagcgtctcctgtggctccatcagctgcttcattctcctttggatcatctggttttaagcctaccctggaaagcacaccagtgccaagtgtgtctgctccaaatatagcaatgaagccctccttcccaccctcaacctctgctgtcaaagtcaaccttagtgaaaagtttactgctgcagctacctctactcctgttagtagctcccagagcgcacccccgatgtcgccattctcttctgcctccaagccagctgcttctggaccactcagccaccccacacctctctcagcaccacctagttccgtgccattgaagtcctcagtcttgccctcaccatcaggacgatctgctcagggcagttcaagcccagtgccctcaatggtacagaaatcacccaggataacccctccagcggcaaagccaggctctccccaggcaaagtcacttcagcctgctgttgcagaaaagcagggacatcagtggaaagattcagatcctgtaatggctggaattggggaggagattgcacactttcagaaggagttggaagagttaaaagcccgaacttccaaagcctgtttccaagtgggcacttctgaggagatgaagatgctgcgaacagaatcagatgacttgcatacctttcttttggagattaaagagaccacagagtcgcttcatggagatataagtagcctgaaaacaactttacttgagggctttgctggtgttgaggaagccagagaacaaaatgaaagaaatcgtgactctggttatctgcatttgctttataaaagaccactggatcccaagagtgaagctcagcttcaggaaattcggcgccttcatcagtatgtgaaatttgctgtccaagatgtgaatgatgttctagacttggagtgggatcagcatctggaacaaaagaaaaaacaaaggcacctgcttgtgccagagcgagagacactgtttaacaccctagccaacaatcgggaaatcatcaaccaacagaggaagaggctgaatcacctggtggatagtcttcagcagctccgcctttacaaacagacttccctgtggagcctgtcctcggctgttccttcccagagcagcattcacagttttgacagtgacctggaaagcctgtgcaatgctttgttgaaaaccaccatagaatctcacaccaaatccttgcccaaagtaccagccaaactgtcccccatgaaacaggcacaactgagaaacttcttggccaagaggaagaccccaccagtgagatccactgctccagccagcctgtctcgatcagcctttctgtctcagagatattatgaagacttggatgaagtcagctcaacgtcatctgtctcccagtctctggagagtgaagatgcacggacgtcctgtaaagatgacgaggcagtggttcaggcccctcggcacgcccccgtggttcgcactccttccatccagcccagtctcttgccccatgcagcaccttttgctaaatctcacctggttcatggttcttcacctggtgtgatgggaacttcagtggctacatctgctagcaaaattattcctcaaggggccgatagcacaatgcttgccacgaaaaccgtgaaacatggtgcacctagtccttcccaccccatctcagccccgcaggcagctgccgcagcagcactcaggcggcagatggccagtcaggcaccagctgtaaacactttgactgaatcaacgttgaagaatgtccctcaagtggtaaatgtgcaggaattgaagaataaccctgcaaccccttctacagccatgggttcttcagtgccctactccacagccaaaacacctcacccagtgttgaccccagtggctgctaaccaagccaagcaggggtctctaataaattcccttaagccatctgggcctacaccagcatccggtcagttatcatctggtgacaaagcttcagggacagccaagatagaaacagctgtgacttcaaccccatctgcttctgggcagttcagcaagcctttctcattttctccatcagggactggctttaattttgggataatcacaccaacaccgtcttctaatttcactgctgcacaaggggcaacaccctccactaaagagtcaagccagccggacgcattctcatctggtgggggaagcaaaccttcttatgaggccattcctgaaagctcacctccctcaggaatcacatccgcatcaaacaccaccccaggagaacctgccgcatctagcagcagacctgtggcaccttctggaactgctctttccaccacctctagtaagctggaaaccccaccgtccaagctgggagagcttctgtttccaagttctttggctggagagactctgggaagtttttcaggactgcgggttggccaagcagatgattctacaaaaccaaccaataaggcttcatccacaagcctaactagtacccagccaaccaagacgtcaggcgtgccctcagggtttaattttactgcccccccggtgttagggaagcacacggagccccctgtgacatcctctgcaaccaccacctcagtagcaccaccagcagccaccagcacttcctcaactgccgtttttggcagtctgccagtcaccagtgcaggatcctctggggtcatcagttttggtgggacatctctaagtgctggcaagactagtttttcatttggaagccaacagaccaatagcacagtgcccccatctgccccaccaccaactacagctgccactccccttccaacatcattccccacattgtcatttggtagcctcctgagttcagcaactaccccctccctgcctatgtccgctggcagaagcacagaagaggccacttcatcagctttgcctgagaagccaggtgacagtgaggtctcagcatcagcagcctcacttctagaggagcaacagtcagcccagcttccccaggctcctccgcaaacttctgactctgttaaaaaagaacctgttcttgcccagcctgcagtcagcaactctggcactgcagcatctagtactagtcttgtagcactttctgcagaggctaccccagccaccacgggggtccctgatgccaggacggaggcagtaccacctgcttcctccttttctgtgcctgggcagactgctgtcacagcagctgctatctcaagtgcaggccctgtggccgtcgaaacatcaagtacccccatagcctccagcaccacgtccattgttgctcccggcccatctgcagaggcagcagcatttggtaccgtcacttctggctcatccgtctttgctcagcctcctgctgccagttctagctcagctttcaaccagctcaccaacaacacagccactgccccctctgccacgcccgtgtttgggcaagtggcagccagcaccgcaccaagtctgtttgggcagcagactggtagcacagccagcacagcagctgccacaccacaggtcagcagctcagggtttagcagcccagcttttggtaccacagccccaggggtctttggacagacaaccttcgggcaggcctcagtctttgggcagtcggcgagcagtgctgcaagtgtcttttccttcagtcagcctgggttcagttccgtgcctgccttcggtcagcctgcttcctccactcccacatccaccagtggaagtgtctttggtgccgcctcaagtaccagtagctccagttccttctcatttggacagtcttctcccaacacaggaggggggctgtttggccaaagcaacgctcctgcttttgggcagagtcctggctttggacagggaggctctgtctttggtggtacctcagctgccaccacaacagcagcaacctctgggttcagcttttgccaagcttcaggttttgggtctagtaatactggttctgtgtttggtcaagcagccagtactggtggaatagtctttggccagcaatcatcctcttccagtggtagcgtgtttgggtctggaaacactggaagagggggaggtttcttcagtggccttggaggaaaacccagtcaggatgcagccaacaaaaacccattcagctcggccagtgggggctttggatccacagctacctcaaatacctctaacctatttggaaacagtggggccaagacatttggtggatttgccagctcgtcgtttggagagcagaaacccactggcactttcagctctggaggaggaagtgtggcatcccaaggctttgggttttcctctccaaacaaaacaggtggcttcggtgctgctccagtgtttggcagccctcctacttttgggggatcccctgggtttggaggggtgccagcattcggttcagccccagcctttacaagccctctgggctcgacgggaggcaaagtgttcggagagggcactgcagctgccagcgcaggaggattcgggtttgggagcagcagcaacaccacatccttcggcacgctcgcgagtcagaatgcccccactttcggatcactgtcccaacagacttctggttttgggacccagagtagcggattctctggttttggatcaggcacaggagggttcagctttgggtcaaataactcgtctgtccagggttttggtggctggcgaagctgagggcgtgtcagcaggcctttcgatccctgggaccaaccgcatcctcagcttcttccccgagaaatgctggagcaggctgttcagaccgacgttgccatcaaaacacatacacccagaaagaaacaacagaaaccaaaactcacaaggcgcatgattacttgttttatatttcatgttgggttttccctcccactattaaacagtctgtttccgtacaaaaaaaaaaaaaa

SEQ ID NO: 27 - DNA, 2717 bp, Homo sapiens
gcgcttccggtgcgacgctgtctctccatgccaggactgagttgtgggggagggaggcggttagcgggctttagcgccttttctggcggcggtagatttgaagcgcttcaaaggaccggacccagagaagaggaaaactctaccggtgcaggagcacagggatcagttgtccttgtttttttttggtcttttcttcatttgaagattaagtattggagccatgggaataaaggttcaacgtcctcgatgtttttttgacattgccattaacaatcaacctgctggaagagttgtctttgaattattttctgatgtgtgccccaaaacatgcgagaactttcgttgtctttgtacaggtgaaaaggggaccgggaaatcaactcagaaaccattacattataagagttgtctctttcacagagttgtcaaggattttatggttcaaggtggtgacttcagtgaaggaaatggacgaggaggggaatctatctatggaggattttttgaagacgagagtttcgctgttaaacacaacaaagaatttctcttgtcaatggccaacagagggaaggatacaaatggttcacagttcttcataacaacgaaaccaactcctcatttagatgggcatcatgttgtttttggacaagtaatctctggtcaagaagttgtaagagagattgaaaaccagaaaacagatgcagctagcaaaccgtttgcggaggtacggatactcagttgtggagagctgattcccaaatctaaagttaagaaagaagaaaagaaaaggcataaatcatcatcatcttcctcctcctcatctagtgactcagatagctcaagtgattctcagtcctcttctgattcctctgattccgaaagtgctactgaagagaaatcaaagaaaagaaaaaagaaacatcggaaaaattcccgaaaacacaagaaagaaaagaaaaagcgaaagaaaagcaagaagagtgcatctagtgagagtgaagctgaaaatcttgaagcacaaccccagtctactgtccgtccagaagagatccctcctatacctgaaaatagattcctaatgagaaaaagtcctcctaaagctgatgagaaggaaaggaaaaacagagagagagaaagggaaagagagtgtaatccacctaactcccagcctgcttcataccagagacgacttttagttactagatctggcaggaaaattaaaggaagaggaccaaggcgttatcgaactccttccagatccagatcaagggatcgtttcagacgtagtgagactcctccacattggaggcaagagatgcagagagctcaaagaatgagggtatcaagtggtgaaagatggatcaagggggataagagtgagttgaatgaaataaaagaaaatcagagaagtccagttagagtaaaagagagaaaaataacagatcacaggaatgtatctgagagtccaaacagaaaaaatgaaaaggagaagaaagttaaagaccataaatctaacagcaaagagagagacatcagaagaaattcagaaaaagatgacaagtataaaaacaaagtgaagaaaagggccaaatctaaaagtaggagtaagagcaaagagaaatcaaagagtaaagaaagagattcaaaacataatagaaatgaagaaaagaggatgaggtcaaggagtaaaggaagggatcatgaaaatgttaaagaaaaagaaaagcagtctgattctaaaggaaaagatcaggaaaggagtagaagtaaagagaagtctaaacagttagaatcaaagagtaatgagcatgatcacagtaaaagtaaggaaaaggatagacgcgcacaatccaggagtagagaatgtgatataactaaaggtaaacacagttataatagcagaacaagagaacgaagcagaagtagggacagaagcagaagagtgcgatcaagaacccatgacagagatcgcagcagaagcaaggagtaccatagatacagagaacaggaatacaggagaagaggacggtcacgaagccgagagagaagaacaccaccaggaagatcaagaagtaaagataggaggagaaggaggagagactcacggagctcagagagagaagaaagtcaaagcagaaacaaagacaaatacagaaaccaagagagtaagagctcacacagaaaagaaaattctgagagtgagaaaagaatgtactctaaaagtcgtgatcataatagctcaaataacagcagggaaaaaaaggctgatagagatcaaagtcccttctcaaaaataaaacaaagcagtcaggacaatgaattaaagtcctccatgttgaaaaataaggaggatgagaagatcagatcctcagtggaaaaagaaaaccaaaaatcaaaaggtcaagaaaatgaccatgtacatgaaaaaaataaaaaatttgatcatgaatcaagccctggaacagatgaagacaaaagcggatgagtgagttatataaacttacttccattctgtttcggattttaagtttgagagacttgctaatgaatctcctttatgttgttttccttttcattgtttttggattgttttatgtttgtccttttttttcttaatgtggatttcattgagttgattttttgataatctgcaatctggataatttgtactgctaaagttttaataaactcgacatgagaaaaacaaaaaaaaaaaa

SEQ ID NO: 28 - DNA, 20 bp, Homo sapiens
actggatcccaagagtgaag

SEQ ID NO: 29 - DNA, 20 bp, Homo sapiens
tcacatcttggacagcaaat